(19) United States
(12) Patent Application Publication
(10) Pub. No.: US 2024/0398786 A1
(43) Pub. Date: Dec. 5, 2024

(54) 7-ETHYL-10-HYDROXY-CAMPTOTHECIN (SN-38) ALBUMIN CONJUGATES FOR TREATMENT OF CANCERS

(71) Applicant: Ohio State Innovation Foundation, Columbus, OH (US)

(72) Inventors: Terence WILLIAMS, Glendora, CA (US); Robert LEE, Lewis Center, OH (US)

(21) Appl. No.: 18/580,878

(22) PCT Filed: Jul. 20, 2022
(86) PCT No.: PCT/US22/73941

§ 371 Date: Jan. 19, 2024

Related U.S. Application Data
(60) Provisional application No. 63/223,797, filed on Jul. 20, 2021.

(51) Int. Cl. A61K 31/4745; A61K 47/64; A61P 35/00

(52) U.S. Cl. CPC A61K 31/4745; A61K 47/643; A61P 35/00

(57) ABSTRACT

The present disclosure provides albumin conjugates for the treatment of cancer, and more particularly conjugates of albumin with 7-ethyl-10-hydroxy-camptothecin.

### CROSS-REFERENCE TO RELATED APPLICATIONS

This application claims the benefit of priority to U.S. Provisional Application No. 63/223,797, filed Jul. 20, 2021, the disclosure of which is incorporated herein by reference in its entirety.

### STATEMENT REGARDING FEDERALLY SPONSORED RESEARCH OR DEVELOPMENT

This invention was made with government support under Grant No. R01 CA198128 awarded by the National Institutes of Health. The Government has certain rights in the invention.

### TECHNICAL FIELD

This disclosure relates to albumin conjugates for the treatment of cancer and, more particularly, to conjugates of albumin with 7-ethyl-10-hydroxy-camptothecin (SN-38).

### BACKGROUND

Human serum albumin (HSA) is the most abundant protein found in human blood. Several properties of HSA make this protein an attractive candidate as a drug carrier in developing novel chemotherapeutics (1,2). Owing to its strong hydrophobic nature, chemical conjugation to this protein allows otherwise insoluble hydrophobic compounds to be dissolved in clinically compatible solvents (3,4). As a robustly stable protein with an unusually long half-life of 19 days, HSA has been reported to extend the pharmacokinetic timeline of compounds bound to it (5,6). Furthermore, it has been well documented that HSA naturally targets tumors, accumulating in abundance at highly proliferative sites within tumors due to the enhanced permeability and retention effect (EPR), in part related to impaired tumor angiogenesis and leaky vascular fenestrae (7-9). In addition to this extracellular tumor localization, HSA is readily internalized by tumor cells via caveolae-mediated endocytosis (6, 10, 11).

Caveolae are flask-shaped invaginations in the plasma membrane. Previous publications have established caveolin-1 (Cav-1) as the principal structural protein of caveolae and, thus, necessary for caveolae-mediated endocytosis (12). Cav-1 is upregulated in many cancer types, including pancreatic and non-small cell lung cancers (13-16). Although tumor-type context dependency has been found, Cav-1 upregulation has been primarily reported to be associated with cancer progression (14,17). It has also been shown previously that Cav-1 expression plays a critical role in mediating albumin uptake and response to albumin-based chemotherapies (11).

Irinotecan, a cytotoxic anticancer agent, is widely used for numerous patients worldwide, including those with non-small cell lung, pancreatic, advanced gastric, and cervical cancer (18). SN-38 (7-ethyl-10-hydroxy-camptothecin), the active metabolite of irinotecan, inhibits DNA topoisomerase I (Top1), disrupting DNA replication and transcription, which results in cell death (19-21). SN-38 is 100-1,000 fold more potent than irinotecan but is virtually insoluble in any pharmaceutical solvent, limiting its clinical application.

There is a clear need for novel therapeutics that may be used in treating cancers, particularly cancers that express caveolin-1.

### SUMMARY

The present disclosure provides a conjugate of SN-38 and an albumin polypeptide and its use in treating medical disorders such as cancer.

Thus, in one aspect, an albumin conjugate is provided comprising an albumin polypeptide covalently bound to one or more groups of Formula I:

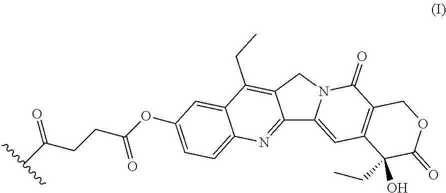 [SMILES: *C(=O)CCC(=O)Oc1ccc2nc3c(c(CC)c2c1)Cn1c-3cc2c(c1=O)COC(=O)[C@]2(O)CC]

wherein [symbol] shows the point of attachment to the albumin polypeptide.

Pharmaceutical compositions are provided comprising a therapeutically effective amount of the albumin conjugates described herein and a pharmaceutically acceptable carrier or excipient.

In another aspect, methods are provided for treating cancer in a subject in need thereof, comprising administering a therapeutically effective amount of the albumin conjugates described herein.

The details of one or more embodiments of the disclosure are set forth in the accompanying drawings and the description below. Other features, objects, and advantages of the disclosure will be apparent from the description, drawings, and claims.

### DESCRIPTION OF DRAWINGS

FIGS. 1A-1D show characterization data of SSH20. (FIG. 1A) SN-38-HSA synthesis scheme. The process consists of SN-38 (Compound 1), SN38-suc (Compound 2), SN38-suc-NHS (Compound 3), and SSH20 (Compound 4). (FIG. 1B) MALDI-TOF mass spectrometry demonstrated an average of 7.75 SN38 molecules per HSA. The linkage between SN38 and HSA was tested by SDS-PAGE/Coomassie stain (FIG. 1C) and organic solvent extraction (FIG. 1D), respectively, showing slower migration of SSH20 compared to free HSA and resistance of SSH20 to organic solvent extraction.

FIGS. 2A-2D shows that Cav-1 expression mediates the uptake of HSA in vitro. (FIG. 2A) Cav-1 expression in stable MIA-PaCa2 (MP2)-shCtrl/shCav-1 and H23shCtrl/shCav-1 cells by immunoblotting. (FIG. 2B) Direct fluorescence images of 1s internalized FITC-labeled HSA demonstrate reduced HSA uptake in stable Cav-1 knockdown MP2/H23 cells. (FIG. 2C) Quantification of internalized FITC-labeled HSA in stable MP2 and H23 cells, reported as the mean of average intensity per cell (with SEM), measured in no less than 50 individual cells for each column. ****, p<0.0001. (FIG. 2D) Uptake and expression of HSA detected by immunoblotting in shCtrl and shCav-1 cells treated with increasing doses of SSH20 for 1 h. GAPDH is shown as equal loading control for A and D.

FIGS. 3A-3H shows that SSH20 targets cancer cells with moderate-high Cav-1 expression in vitro. (FIG. 3A and FIG. 3B) Direct fluorescence images of internalized SN-38 in shCtrl and shCav-1 cells pulsed with 250 nM of either SN-38/SSH20 for 2 h prior to cell fixation. (FIG. 3C and FIG. 3D) Quantitation of internalized SN-38 in shCtrl and shCav-1 cells. Quantitation is reported as the mean average intensity per cell (with SEM), measured in no less than 50 individual cells for each column. ****, p<0.0001; n.s., p>0.05. (FIG. 3E and FIG. 3F) Representative fluorescence images of Cav-1 expression (red) and internalized SN-38 (green) in shCtrl and shCav-1 cells, co-cultured at a 1:1 ratio and treated with 250 nM SSH20 for 2 h prior to fixation. (FIG. 3G and FIG. 3H) Correlation between Cav-1 intensity and SN-38 intensity in co-cultured shCtrl and shCav-1 cells. Each data point represents an individual cell. No less than 50 cells were measured to generate Pearson correlation plots. The line of best fit is indicated by the red line with 95% confidence bands (dashed lines). P and R² values were reported.

FIG. 4A-4D show that Cav-1 depletion reduces sensitivity to SSH20 in vitro. (FIG. 4A and FIG. 4B) Cell cytotoxicity was determined in MP2-shCtrl/shCav-1 and H23-shCtrl/shCav-1 stable cells with different doses of SN-38 and SSH20 for different periods of drug exposure (6, 12, 72 h), using AlamarBlue assay. ***, p<0.001; **, p<0.01; *, p<0.05. (FIG. 4C) IC50 of each treatment group was calculated and compared in MP2-shCtrl/shCav-1 and H23-shCtrl/shCav-1 cells. Fold change (IC50 shCav-1/IC50 shCtrl) shows an increasing fold-change difference with shorter time periods of exposure. (FIG. 4D) SSH20-induced apoptosis was confirmed by immunoblotting for cleaved PARP (cPARP) and cleaved caspase 3 (cCas3). GAPDH is shown as an equal loading control.

FIGS. 5A-5E show the high sensitivity and potency of SSH20 in tumors with moderate-high Cav-1 expression in vivo. (FIG. 5A) Experimental treatment schema. MP2-shCtrl/shCav-1 and H23-shCtrl/shCav-1 cells were injected into the flanks of mice. Once tumors reached approximately 100-200 mm3 in size, 0.9% saline (vehicle), SSH20 (10 mg/kg), or the molar equivalent of irinotecan was administered intravenously via retro-orbital injection. The tumor growth rate was significantly reduced in the SSH20-treated groups compared to the control and irinotecan groups in MP2-shCtrl and H23-shCtrl groups (FIG. 5B and FIG. 5D), but not in MP2-shCav-1 and H23-shCav-1 groups (FIG. 5C and FIG. 5E). Similarly, tumor doubling time was significantly prolonged in SSH20 treated groups compared to the control and irinotecan groups in MP2-shCtrl and H23-shCtrl groups (FIG. 5B and FIG. 5D), but not in MP2-shCav-1 and H23-shCav-1 groups (FIG. 5C and FIG. 5E). n=10/group, *, p<0.01.

FIG. 6 shows that SSH20 treatment results in no detectable changes in weight compared to vehicle-treated mice. H23-shCtrl/shCav-1 cells were injected into the flanks of mice. Once tumors reached approximately 100-200 mm3 in size, 0.9% saline (vehicle), or SSH20 (5 or 10 mg/kg) was administered intravenously via retro-orbital injection. No significant differences in weight were detected between vehicle or SSH20 treated groups.

FIG. 7 shows the kinetics of SN-38 release from SSH20 incubated in human serum. The half-life was calculated by one-phase exponential association. The release of SN-38 from SSH20 followed a time-dependent manner with a half-life of ˜64.2 hours.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features that may be readily separated from or combined with the features of any of the other several embodiments without departing from the scope or spirit of the present disclosure.

Any recited method can be carried out in the order of events recited or in any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning an arrangement of steps or operational flow, plain meaning derived from grammatical organization or punctuation, or the number or type of aspects described in the specification.

All publications mentioned herein are incorporated herein by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise. Thus, for example, reference to “a cancer,” “a compound,” or “a cell” includes, but is not limited to, two or more such cancers, compounds, or cells, and the like.

It should be noted that ratios, concentrations, amounts, and other numerical data can be expressed herein in a range format. It can be further understood that the endpoints of each of the ranges are significant both in relation to and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, using the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x,’ ‘about y’, and ‘about z’ as well as the ranges of ‘less than x,’ less than y’, and ‘less than z.’ Likewise, the phrase ‘about x, y, z, or greater’ should be interpreted to include the specific ranges of ‘about x,’ ‘about y’, and ‘about z’ as well as the ranges of ‘greater than x,’ greater than y’, and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate, larger, or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. It is understood that where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be to delay or even prevent the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single dose compositions can contain such amounts or submultiples thereof to make up the daily dose. The individual physician can adjust the dosage in the event of any contraindications. It is generally preferred that a maximum dose of the pharmacological agents of the invention (alone or in combination with other therapeutic agents) be used, that is, the highest safe dose according to sound medical judgment. However, those of ordinary skill in the art will understand that a patient may insist upon a lower or tolerable dose for medical, psychological, or virtually any other reasons.

A response to a therapeutically effective dose of a disclosed composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The treatment amount may vary, for example, by increasing or decreasing the amount of a disclosed compound or pharmaceutical composition, changing the disclosed compound and/or pharmaceutical composition administered, changing the route of administration, changing the dosage timing, and so on. Dosage can vary and can be administered in one or more doses daily for one or several days. Guidance can be found in the literature for appropriate dosages for given classes of pharmaceutical products.

As used herein, the term “prophylactically effective amount” refers to an amount effective for preventing the onset or initiation of a disease or condition.

As used herein, “prevent” or “preventing” refers to precluding, averting, obviating, forestalling, stopping, or hindering something from happening, especially by advance action. It is understood that where reduce, inhibit or prevent are used herein unless specifically indicated otherwise, the use of the other two words is also expressly disclosed.

As used herein, the terms “optional” or “optionally” mean that the subsequently described event or circumstance can or cannot occur, and that the description includes instances where said event or circumstance occurs and where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological and/or physiological effect. The effect can be but does not necessarily have to be prophylactic in preventing or partially preventing a disease, symptom, or condition such as cancer. The effect can be therapeutic in terms of a partial or complete cure of a disease, condition, symptom, or adverse effect attributed to the disease, disorder, or condition. The term “treatment,” as used herein, can include any treatment of ophthalmological disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to therapeutic, prophylactic, or therapeutic and prophylactic treatment. Those in need of treatment (subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates, and other isomers, such as rotamers, as if each is described explicitly unless otherwise indicated or otherwise excluded by context. It is to be understood that the compounds provided herein may contain chiral centers. Such chiral centers may be of either the (R—) or (S—) configuration. The compounds herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to the administration of the compound in its (S—) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable salt” is a derivative of the disclosed compound in which the parent compound is modified by making inorganic and organic, pharmaceutically acceptable, acid or base addition salts. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like) or by reacting free base forms of these compounds with a stoichiometric amount of the appropriate acid. Such reactions are typically carried out in water, an organic solvent, or a mixture of the two. Generally, where practicable, non-aqueous media like ether, ethyl acetate, ethanol, isopropanol, or acetonitrile are typical. Salts of the present compounds further include solvates of the compounds and the compound salts. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include salts acceptable for human consumption and the quaternary ammonium salts of the parent compound formed, for example, from inorganic or organic salts. Examples of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high-performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modem methods for purifying compounds to produce substantially chemically pure compounds are known to those of skill in the art. However, a substantially chemically pure compound may be a mixture of stereoisomers.

A “control” is an alternative subject or sample used in an experiment for comparison purposes. A control can be “positive” or “negative.” Various controls within the scope of the present invention are described in more detail below.

“Identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to or may be applied to the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably, over a region that is 10-50 amino acids or 20-50 nucleotides in length. As used herein, percent (%) amino acid sequence identity is defined as the percentage of amino acids in a candidate sequence that is identical to the amino acids in a reference sequence after aligning the sequences and introducing gaps, if necessary, to achieve the maximum percent sequence identity. Alignment for determining percent sequence identity can be achieved in various ways within the skill in the art, for instance, using publicly available computer software such as BLAST, BLAST-2, ALIGN, ALIGN-2, or Megalign (DNASTAR) software. Appropriate parameters for measuring alignment, including any algorithms needed to achieve maximal alignment over the full length of the sequences being compared, can be determined by known methods.

For sequence comparisons, typically, one sequence acts as a reference sequence to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence based on the program parameters.

One example of an algorithm suitable for determining percent sequence identity and sequence similarity is the BLAST and BLAST 2.0 algorithms, described in Altschul et al. (1977) Nuc. Acids Res. 25:3389-3402, and Altschul et al. (1990)J. Mol. Biol. 215:403-410, respectively. Software for performing BLAST analyses is publicly available through the National Center for Biotechnology Information. This algorithm first identifies high-scoring sequence pairs (HSPs) by identifying short words of length W in the query sequence, which either match or satisfy some positive-valued threshold score T when aligned with a word of the same length in a database sequence. T is the neighborhood word score threshold (Altschul et al. (1990)J. Mol. Biol. 215:403-410). This initial neighborhood word hits act as seeds for initiating searches to find longer HSPs containing them. The word hits are extended in both directions along each sequence as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4, and a comparison of both strands. For amino acid sequences, the BLASTP program uses as defaults a word length of 3, an expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which indicates the probability by which a match between two nucleotide or amino acid sequences would occur by chance. For example, a nucleic acid is considered similar to a reference sequence if the smallest sum probability in comparing the test nucleic acid to the reference nucleic acid is less than about 0.2 or less than about 0.01.

A polynucleotide, or fragment thereof, is “operably linked” when placed into a functional relationship with another nucleic acid sequence. For example, DNA encoding an amino-acid presequence or secretory leader is operably linked to DNA encoding a polypeptide if it is expressed as a preprotein that participates in the secretion of the polypeptide; a promoter or enhancer is operably linked to a DNA sequence encoding a polypeptide (also known as a coding sequence) if it affects the transcription of the coding sequence, or a ribosome binding site is operably linked to a coding sequence if it is positioned to facilitate translation. Generally, “operably linked” means that the linked polynucleotide sequences, or fragments thereof, are spatially near each other and, in the case of a secretory leader, contiguous and in the same reading frame. However, operably linked polynucleotides (e.g., enhancers and coding sequences) do not have to be contiguous. Linking can be accomplished, for example, by ligation at convenient restriction sites. If such sites do not exist, synthetic oligonucleotide adaptors or linkers can be used per conventional practice. In some embodiments, a promoter is operably linked with a coding sequence when it is capable of affecting (e.g., modulating relative to the absence of the promoter) the expression of a polypeptide from that coding sequence (e.g., the coding sequence is under the transcriptional control of the promoter).

“Peptide,” “protein,” and “polypeptide” are used interchangeably to refer to a natural or synthetic molecule comprising two or more amino acids linked by the carboxyl group of one amino acid to the alpha amino group of another. The amino acids may be natural or synthetic. They can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance, molecules required for function. Examples of molecules that may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is no requirement that a polypeptide must contain an intended function; a polypeptide can be functional, non-functional, function for unexpected/unintended purposes, or have an unknown function. A polypeptide is comprised of approximately twenty standard naturally occurring amino acids. However, natural and synthetic amino acids, which are not members of the standard twenty amino acids, may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

### Albumin Conjugates

The present disclosure provides albumin polypeptides conjugated to one or more groups having a moiety corresponding to 7-ethyl-10-hydroxy-camptothecin (SN-38):

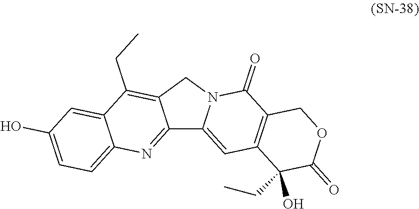

Thus, in one aspect, an albumin conjugate is provided comprising an albumin polypeptide covalently bound to one or more groups of Formula I:

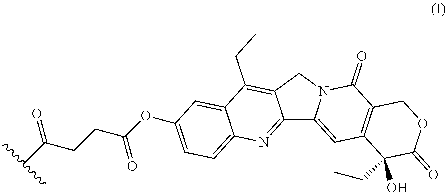 [SMILES: *C(=O)CCC(=O)Oc1ccc2nc3c(c(CC)c2c1)Cn1c-3cc2c(c1=O)COC(=O)[C@]2(O)CC]

wherein [symbol] shows the point of attachment to the albumin polypeptide.

In some embodiments, the albumin polypeptide is covalently bound from 3 to 20 groups of Formula I, for example 3, 4, 5, 6, 7, 8, 9, 10, 11, 12, 13, 14, 15, 16, 17, 18, 19, or 20 groups. In typical embodiments, the albumin polypeptide is covalently bound to 7 or 8 groups of Formula I.

In typical embodiments, the albumin polypeptide comprises a member of the serum albumin family of proteins. Serum albumins are albumins typically found in vertebrate blood. Serum albumins are produced in the liver, occur in blood plasma, and are typically mammals' most abundant blood protein. Serum albumins are typically globular, water-soluble, un-glycosylated serum proteins of approximate molecular weight of 65,000 Daltons. Their general structure is characterized by several long alpha helices allowing them to maintain a relatively static shape. Albumins are negatively charged when ionized in water at pH 7.4 as found in the body.

In some embodiments, the albumin polypeptide comprises bovine serum albumin. In other embodiment, the albumin polypeptide comprises human serum albumin (HSA).

The albumin polypeptide may be a synthetic polypeptide or a natural polypeptide from a species which expresses albumin.

“Albumin,” as used herein, refers to polypeptide comprising albumin, also known as serum albumin, PRO0883, PRO0903, PRO1341, ALB, or HSA. In some embodiments, the albumin polypeptide can be identified in one or more publicly available databases as follows: HGNC: 399 Entrez Gene: 213 Ensembl: ENSG00000163631 OMIM: 103600 UniProtKB: P02768.

In some embodiments, the albumin polypeptide is a polypeptide comprising an amino acid sequence which is at least 70/6 identical to SEQ ID NO: 1. In some embodiments, the albumin polypeptide is a polypeptide comprising an amino acid sequence which is at least 75%, at least 80%, at least 85%, at least 90%, at least 95%, at least 98%, or at least 99% identical to SEQ ID NO: 1. In some embodiments, the albumin polypeptide is a polypeptide comprising SEQ ID NO: 1.

SEQ ID NO: 1
MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

The albumin polypeptide can be encoded by an albumin-encoding polynucleotide. In humans, albumin polypeptide is encoded by the ALB gene. In some embodiments, the albumin polypeptide is encoded by a polynucleotide comprising a nucleic acid sequence which is at least 70% identical to SEQ ID NO: 2. In some embodiments, the albumin polypeptide is encoded by a polynucleotide comprising a nucleic acid sequence which is at least 70%, at least 75%, at least 80%, at least 85%, at least 90%, at least 95%, at least 98%, or at least 99% identical to SEQ ID NO: 2. In some embodiments, the albumin polypeptide is encoded by a polynucleotide comprising the nucleic acid sequence of SEQ ID NO: 2.

SEQ ID NO: 2
AGCTTTTCTCTTCTGTCAACCCCACACGCCTTTGGCACAATGAAGTGGGTAACCT
TTATTTCCCTTCTTTTTCTCTTTAGCTCGGCTTATTCCAGGGGTGTGTTTCGTCGA
GATGCACACAAGAGTGAGGTTGCTCATCGGTTTAAAGATTTGGGAGAAGAAAA
TTTCAAAGCCTTGGTGTTGATTGCCTTTGCTCAGTATCTTCAGCAGTGTCCATTT
GAAGATCATGTAAAATTAGTGAATGAAGTAACTGAATTTGCAAAAACATGTGTT
GCTGATGAGTCAGCTGAAAATTGTGACAAATCACTTCATACCCTTTTTGGAGAC
AAATTATGCACAGTTGCAACTCTTCGTGAAACCTATGGTGAAATGGCTGACTGC
TGTGCAAAACAAGAACCTGGGAGAAATGAATGCTTCTTGCAACACAAAGATGA
CAACCCAAACCTCCCCCGATTGGTGAGACCAGAGGTTGATGTGATGTGCACTGC
TTTTCATGACAATGAAGAGACATTTTTGAAAAAATACTTATATGAAATTGCCAG
AAGACATCCTTACTTTTATGCCCCGGAACTCCTTTTCTTTGCTAAAAGGTATAAA
GCTGCTTTTACAGAATGTTGCCAAGCTGCTGATAAAGCTGCCTGCCTGTTGCCA
AAGCTCGATGAACTTCGGGATGAAGGGAAGGCTTCGTCTGCCAAACAGAGACT
CAAGTGTGCCAGTCTCCAAAAATTTGGAGAAAGAGCTTTCAAAGCATGGGCAG
TAGCTCGCCTGAGCCAGAGATTTCCCAAAGCTGAGTTTGCAGAAGTTTCCAAGT
TAGTGACAGATCTTACCAAAGTCCACACGGAATGCTGCCATGGAGATCTGCTTG
AATGTGCTGATGACAGGGCGGACCTTGCCAAGTATATCTGTGAAAATCAAGATT
CGATCTCCAGTAAACTGAAGGAATGCTGTGAAAAACCTCTGTTGGAAAAATCCC
ACTGCATTGCCGAAGTGGAAAATGATGAGATGCCTGCTGACTTGCCTTCATTAG
CTGCTGATTTTGTTGAAAGTAAGGATGTTTGCAAAAACTATGCTGAGGCAAAGG
ATGTCTTCTTGGGCATGTTTTTGTATGAATATGCAAGAAGGCATCCTGATTACTC
TGTCGTGCTGCTGCTGAGACTTGCCAAGACATATGAAACCACTCTAGAGAAGTG
CTGTGCCGCTGCAGATCCTCATGAATGCTATGCCAAAGTGTTCGATGAATTTAA
ACCTCTTGTGGAAGAGCCTCAGAATTTAATCAAACAAAATTGTGAGCTTTTTGA
GCAGCTTGGAGAGTACAAATTCCAGAATGCGCTGTTAGTTCGTTACACCAAGAA
AGTACCCGAAGTGTCAACTCCAACTCTTGTAGAGGTCTCAAGAAACCTAGGAA
AAGTGGGCAGCAAATGTTGTAAACATCCTGAAGCAAAAAGAATGCCCTGTGCA
GAAGACTATCTATCCGTGGTCCTGAACCAGTTATGTGTGTTGCATGAGAAAACG
CCAGTAAGTGACAGAGTCACCAAATGCTGCACAGAATCCTTGGTGAACAGGCG
ACCATGCTTTTCAGCTCTGGAAGTCGATGAAACATACGTTCCCAAAGAGTTTAA
TGCTGAAACATTCACCTTCCATGCAGATATATGCACACTTTCTGAGAAGGAGAG
ACAAATCAAGAAACAAACTGCACTTGTTGAGCTCGTGAAACACAAGCCCAAGG
CAACAAAAGAGCAACTGAAAGCTGTTATGGATGATTTCGCTGCTTTTGTAGAGA
AGTGCTGCAAGGCTGACGATAAGGAGACCTGCTTTGCCGAGGAGGGTAAAAAA
CTTGTTGCTGCAAGTCAAGCTGCCTTAGGCTTATAACATCACATTTAAAAGCAT
CTCAGCCTACCATGAGAATAAGAGAAAGAAAATGAAGATCAAAAGCTTATTCA
TCTGTTTTTCTTTTTCGTTGGTGTAAAGCCAACACCCTGTCTAAAAAACATAAAT
TTCTTTAATCATTTTGCCTCTTTTCTCTGTGCTTCAATTAATAAAAAATGGAAAG
AATCT

In some embodiments, the albumin polypeptide comprises one or more lysine residues. In some embodiments, the albumin polypeptide comprises at least two, three, four, five, ten, or fifteen lysine residues. Lysine residues can serve as a point of covalent attachment for the one or more groups of Formula I present in the albumin conjugate.

The present disclosure also includes compounds with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P ³²P, ³⁵S, ³⁶Cl, and ¹²⁵I, respectively. In one embodiment, isotopically labeled compounds can be used in metabolic studies (with ¹⁴C), reaction kinetic studies (with, for example, ²H or ³H), detection or imaging techniques, such as positron emission tomography (PET), or single-photon emission computed tomography (SPECT) including drug and substrate tissue distribution assays, or in radioactive treatment of patients. In particular, an ¹⁸F-labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, such as deuterium (2H) and tritium (3H), may be used anywhere in described structures that achieve the desired result. Alternatively, or in addition, isotopes of carbon, e.g., ¹³C and ¹⁴C, may be used. In one embodiment, the isotopic substitution is replacing hydrogen with deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in the allocation of bond breakage during metabolism (an alpha-deuterium kinetic isotope effect) or next to or near the site of bond breakage (a beta-deuterium kinetic isotope effect).

Isotopic substitutions, for example, deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at the desired location. Unless otherwise stated, the enrichment at any point is above natural abundance and, in an embodiment, is enough to alter a detectable property of the compounds as a drug in a human.

The compounds of the present disclosure may form a solvate with solvents (including water). Therefore, the invention includes a solvated form of the active compound in one embodiment. The term “solvate” refers to a molecular complex of a compound of the present invention (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates per the invention include those wherein the solvent of crystallization may be isotopically substituted, e.g., D₂O, d6-acetone, or d6-DMSO. A solvate can be in a liquid or solid form.

A “prodrug,” as used herein, means a compound that, when administered to a host in vivo, is converted into a parent drug. As used herein, the term “parent drug” means any presently described compound herein. Prodrugs can be used to achieve any desired effect, including enhancing the properties of the parent drug or improving the pharmaceutic or pharmacokinetic properties of the parent, including increasing the drug's half-life in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided with several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compounds that have at least two functional groups that can link the parent compound with another prodrug moiety and are typically biodegradable in vivo. In some embodiments, 2, 3, 4, or 5 biodegradable prodrug moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched, or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art, including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration or at continuous and distinct intervals, as can be readily determined by a person skilled in the art.

Compositions, as described herein, comprising an active compound and a pharmaceutically acceptable carrier or excipient, may be useful in various medical and non-medical applications. For example, pharmaceutical compositions comprising an active compound and an excipient may be useful for treating or preventing a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary or human pharmaceutical or therapeutic use. The terms “carrier” or “pharmaceutically acceptable carrier” can include, but are not limited to, phosphate buffered saline solution, water, emulsions (such as an oil/water or water/oil emulsion), or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any solvents, diluents or other liquid vehicles, dispersion or suspension aids, surface active agents, isotonic agents, thickening or emulsifying agents, preservatives, solid binders, lubricants, and the like, as suited to the particular dosage form desired. General considerations in the formulation or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid, or liquid filler, diluent, encapsulating material, or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt, gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical or cosmetic composition, the choice of the excipient will depend on the route of administration, the agent being delivered, the time course of delivery, etc. It can be administered to humans or animals orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate-controlling membrane or dispersing the particles in a polymer matrix or gel.

The active ingredient may be administered in such amounts, time, and route deemed necessary to achieve the desired result. The exact amount of the active ingredient will vary from subject to subject, depending on the subject's species, age, and general condition, the severity of the medical disorder, the particular active ingredient, its mode of administration, its mode of activity, and the like. The active ingredient, whether the active compound itself or the active compound in combination with an agent, is preferably formulated in dosage unit form for ease of administration and uniformity of dosage. It will be understood, however, that the attending physician will decide the total daily usage of the active ingredient within the scope of sound medical judgment. The specific therapeutically effective dose level for any particular subject will depend upon a variety of factors, including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, or drops), mucosal, nasal, buccal, enteral, sublingual; by intratracheal instillation, bronchial instillation, or inhalation; or as an oral spray, nasal spray, or aerosol. In general, the most appropriate route of administration will depend upon a variety of factors, including the nature of the active ingredient (e.g., its stability in the environment of the gastrointestinal tract), the condition of the subject (e.g., whether the subject can tolerate oral administration), etc.

The exact amount of an active ingredient required to achieve a therapeutically or prophylactically effective amount will vary from subject to subject, depending on species, age, general condition of a subject, severity of the side effects or disorder, identity of the particular compound(s), mode of administration, and the like. The amount to be administered to, for example, a child or an adolescent can be determined by a medical practitioner or person skilled in the art and can be lower, or the same as that administered to an adult.

Useful dosages of the active agents and pharmaceutical compositions disclosed herein can be determined by comparing their in vitro and in vivo activity in animal models. Methods for extrapolating effective dosages in mice, and other animals, to humans are known to the art.

The dosage ranges for administering the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one of skill in the art. The individual physician can adjust the dosage in the event of any counterindications. Dosage can vary and can be administered in one or more doses daily for one or several days.

### Methods of Treatment

The present disclosure also provides methods for treating or preventing cancer in a subject, comprising administering to the subject a therapeutically effective amount of a compound or composition disclosed herein. The methods can further comprise administering one or more additional therapeutic agents, such as anti-cancer or anti-inflammatory agents. Additionally, the method can further comprise administering a therapeutically effective amount of ionizing radiation to the subject.

Methods of killing a cancer or tumor cell are also provided, comprising contacting the cancer or tumor cell with an effective amount of a compound or composition as described herein. The methods can further include administering one or more additional therapeutic agents or an effective amount of ionizing radiation.

The disclosed methods can optionally include identifying a patient who is or can need treatment of an oncological disorder. The patient can be a human or other mammal, such as a primate (monkey, chimpanzee, ape, etc.), dog, cat, cow, pig, or horse, or other animals having an oncological disorder. In some aspects, the subject can receive the therapeutic compositions before, during, or after surgical intervention to remove part or all of a tumor.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent or an assimilable edible carrier for oral delivery. In addition, the active compound can be incorporated into sustained-release preparations or devices.

For the treatment of an oncological disorder, compounds, agents, and compositions disclosed herein can be administered to a patient in need of treatment before, after, or in combination with other antitumor or anticancer agents or substances (e.g., chemotherapeutic agents, immunotherapeutic agents, radiotherapeutic agents, cytotoxic agents, etc.) or with radiation therapy or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, or other anti-cancer drugs or antibodies, such as, for example, imatinid or trastuzumab. These other substances or radiation treatments can be given simultaneously or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein before, after, or in combination with the administration of a chemotherapeutic agent, an immunotherapeutic agent, a radiotherapeutic agent, or radiotherapy.

The term “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show a partial or complete lack of structural organization and functional coordination with normal tissue. Most invade surrounding tissues, can metastasize to several sites, are likely to recur after attempted removal and may cause the patient's death unless adequately treated. As used herein, “neoplasia” describes all cancerous disease states and embraces or encompasses the pathological process associated with malignant, hematogenous, ascitic, and solid tumors. The cancers treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstrom macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer: hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

In particular embodiments, the cancer may be selected from melanoma, mesothelioma, a sarcoma, pancreatic, lung, breast, head and neck, urothelial, bladder, kidney, esophageal, and prostate cancer.

Also disclosed are methods of treating cancers associated with Cav-1 expression or levels. In some embodiments, the cancer to be treated expresses Cav-1. In further embodiments, the cancer to be treated has elevated levels of Cav-1 compared to a control.

In one aspect, a method is provided for treating a cancer in a subject in need thereof comprising:
a) determining whether the cancer expresses caveolin 1 (Cav-1); and
b) if the cancer is determined to express Cav-1 in a), administering a therapeutically effective amount of an albumin conjugate described herein.

In another aspect, a method of treating a cancer in a subject in need thereof comprising:
a) determining whether cancer expresses an increased amount of caveolin 1 (Cav-1) compared to a control, wherein the control comprises a non-cancerous cell of a same tissue type as the cancer; and
b) if the cancer is determined to express an increased amount of Cav-1 in a), administering a therapeutically effective amount of an albumin conjugate described herein.

Also disclosed are methods of selecting a subject for treatment with the herein disclosed compounds or pharmaceutical compositions. The methods can include obtaining a biological sample of the subject. In some embodiments, a biological sample can be obtained from a subject or from another biological sample of a subject in which the subject is suspected of having a tumor or cancer or is known to have a tumor or cancer. The biological sample typically contains cells suspected or known to be tumorous or cancerous or should contain biological material (e.g., extracted polynucleotides or polypeptides) from such cells. The biological ample can be obtained by any suitable method for further analysis (e.g., measuring Cav-1 levels). For example, the sample can be obtained by tissue scraping, biopsy (e.g., surgical biopsy, fine-needle aspiration biopsy, core needle biopsy, stereotactic biopsy, etc.), phlebotomy techniques, or other suitable methods.

The methods include determining a level of Cav-1 in the biological sample. In some embodiments, the methods can also include determining a level of Cav-1 in a control. Cav-1 levels in both the biological sample and the control can be determined via various methods used to determine polynucleotide or polypeptide levels. As used herein, the “level” of a polynucleotide or polypeptide can refer to an expression level, a functionality level, or combinations thereof. The biological sample or a portion thereof may be further processed according to standard protocols for the method selected to determine the polynucleotide or polypeptide levels.

For example, a Cav-1 level can be determined as a level of Cav-1 polypeptide expression, which refers to a qualitative or quantitative amount of polypeptide within the biological sample or control, for example, within the cells or cell lysates of the biological sample or control. Polypeptide expression levels can be determined by a number of methods, including radiation absorbance (e.g., ultraviolet light absorption at 260, 280, or 230 nm), bicinchoninic acid (BCA) assay, Bradford assay, biuret test, Lowry method, Coomassie-blue staining, silver-staining, immunodetection and/or Western blot analysis, or other suitable methods.

Optionally, a Cav-1 level can be determined as a level of Cav-1 polynucleotide expression, which refers to a qualitative or quantitative amount of RNA polynucleotide within the sample or control, for example, within the cells of the sample or control. A number of methods can determine polynucleotide expression levels, such as mRNA transcript levels, including radiation absorbance (e.g., ultraviolet light absorption at 260, 280, or 230 nm), quantification of fluorescent dye or tag emission (e.g., ethidium bromide intercalation), quantitative polymerase chain reaction (qPCR) of cDNA produced from mRNA transcripts, southern blot analysis, gene expression microarray, or other suitable methods. Levels of mRNA transcripts can also be used to infer or estimate levels of polypeptide expression.

Optionally, a Cav-1 level can be determined as a level of Cav1 polypeptide functionality, which refers to qualitative or quantitative measurement of Cav-1 polypeptide's performance of any one or more functions known to be associated with Cav-1 polypeptide. Alternatively or in addition, the term “Cav-1 polypeptide functionality” can also refer to a qualitative or quantitative measurement of Cav-1 polypeptide's physical state (e.g., polypeptide folding, accessibility, or mutation) known to affect Cav-1 polypeptide's performance of any one or more functions known to be associated with Cav-1 polypeptide. Thus, while a polypeptide expression level may or may not be altered compared to a control, the function of the polypeptide can be reduced compared to a control. Cav-1 polypeptide functionality can be determined by, for example, and without limitation, secondary or tertiary folding analysis (e.g., incomplete or incorrect protein folding determinable by circular dichroism, crystallography, nuclear magnetic resonance, electron microscopy, protein folding prediction programs, or other methods), sequestration experiments (e.g., coimmunoprecipitation with a repressor or inhibitor) compartmentalization experiments (e.g., microscopy observed cellular localization), functional or enzymatic assay (e.g., caveolae-binding assay), presence of amino acid sequence mutations known to reduce function, or other suitable methods.

Optionally, a Cav-1 level can be determined as a level of Cav-1 polynucleotide functionality, which refers to qualitative or quantitative measurement of Cav-1 polynucleotide's performance of any one or more functions known to be associated with Cav-1 polynucleotide. Alternatively or in addition, the term “Cav1 polynucleotide functionality” can also refer to a qualitative or quantitative measurement of Cav-1 polynucleotide's physical state (e.g., polynucleotide folding, accessibility, or mutation) known to affect Cav-1 polynucleotide's performance of any one or more functions known to be associated with Cav-1 polynucleotide, or the downstream Cav-1 polypeptide's performance of any one or more functions known to be associated with Cav-1 polypeptide. Thus, while a polynucleotide expression level may or may not be altered compared to a control, the function of the polynucleotide can be reduced compared to a control. Cav-1 polynucleotide functionality can be determined by, for example, and without limitation, mRNA folding analysis (e.g., inhibitory hairpin formation determinable by nucleotide accessibility experiments, mRNA folding prediction programs, or other methods), DNA modification experiments (e.g., DNA-transcription promoter/repressor binding assays, histone modification assays, methylation analysis) functional or enzymatic assay (e.g., mRNA translation assays), presence of nucleic acid sequence mutations known to reduce function, or other suitable methods.

The determined Cav-1 levels can be compared to a control. In some embodiments, the Cav-1 levels in the biological sample are readily detectable and thus sufficiently increased compared to a control to positively select the subject for treatment with any of the herein disclosed compounds or pharmaceutical compositions. This is because Cav-1 is undetectable or very minimally expressed in some tissues. In some embodiments, the Cav-1 levels in the biological sample are at least 10% increased compared to a control. In some embodiments, the Cav1 levels in the biological sample are at least 50%, at least 75%, at least 100%, at least 150%, at least 200%, at least 250%, or at least 500% increased compared to a control.

The control can comprise a biological sample or values obtained from analyzing the control sample or a collection of values used as a standard applied to one or more subjects (e.g., a general number or average known and not identified in the method using a sample). In some embodiments, the control can comprise a biological sample of the subject known not to be or suspected not to be cancerous (e.g., a baseline sample). In some embodiments, the control can comprise the subject's non-cancerous cells, which are of the same cell type as the cells of the biological sample. Typically, the level of Cav-1 is determined in a control without additional steps or manipulations performed on the control beyond those required to obtain the control and determine Cav-1 levels. However, additional storage steps (e.g., in cryogenic conditions), washing steps (e.g., in buffered solutions), and other steps not expected to significantly affect the results upon determining Cav1 levels can be included for both the control and the biological sample.

An increased level of Cav-1 can indicate that the subject will respond therapeutically to a treatment comprising administering any of the herein disclosed compounds or pharmaceutical compositions. Thus, the methods can further comprise administering any one or more of the herein disclosed compounds or pharmaceutical compositions to a subject having an increased level of Cav-1 (e.g., in comparison to a control). Alternatively, the methods can further comprise not administering any of the herein disclosed compounds or pharmaceutical compositions to a subject having a level of Cav-1 that is not increased or is decreased (e.g., in comparison to a control).

Further embodiments of the present invention are provided as follows:

Embodiment 1. An albumin conjugate comprising an albumin polypeptide covalently bound to one or more groups of Formula I:

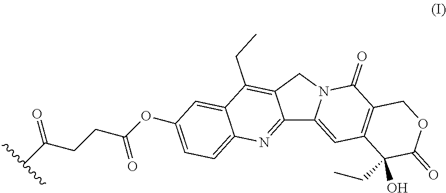 [SMILES: *C(=O)CCC(=O)Oc1ccc2nc3c(c(CC)c2c1)Cn1c-3cc2c(c1=O)COC(=O)[C@]2(O)CC]

wherein [symbol] shows the point of attachment to the albumin polypeptide.

Embodiment 2. The albumin conjugate of embodiment 1, wherein the albumin polypeptide is covalently bound to from 3 to 20 groups of Formula I.

Embodiment 3. The albumin conjugate of embodiment 1 or 2, wherein the albumin polypeptide is covalently bound to 7 or 8 groups of Formula I.

Embodiment 4. The albumin conjugate of any one of embodiments 1-3, wherein the albumin polypeptide comprises one or more lysine residues.

Embodiment 5. The albumin conjugate of any one of embodiments 1-3, wherein the albumin polypeptide comprises at least two, three, four, five, ten, or fifteen lysine residues.

Embodiment 6. The albumin conjugate of any one of embodiments 1-3, wherein the albumin polypeptide comprises human serum albumin.

Embodiment 7. The albumin conjugate of any one of embodiments 1-3, wherein the albumin polypeptide comprises bovine serum albumin.

Embodiment 8. The albumin conjugate of any one of embodiments 1-3, wherein the albumin polypeptide is a polypeptide comprising an amino acid sequence which is at least 70% identical to SEQ ID NO: 1.

Embodiment 9. The albumin conjugate of embodiment 8, wherein the albumin polypeptide is a polypeptide comprising an amino acid sequence which is at least 75%, at least 80%, at least 85%, at least 90%, at least 95%, at least 98%, or at least 99% identical to SEQ ID NO: 1.

Embodiment 10. The albumin conjugate of embodiment 8 or 9, wherein the albumin polypeptide is a polypeptide comprising SEQ ID NO: 1.

Embodiment 11. A pharmaceutical composition comprising a therapeutically effective amount of the albumin conjugate of any one of embodiments 1-10 and a pharmaceutically acceptable carrier or excipient.

Embodiment 12. A method of treating a cancer in a subject in need thereof comprising administering a therapeutically effective amount of an albumin conjugate of any one of embodiments 1-10 or a pharmaceutical composition of embodiment 11.

Embodiment 13. The method of embodiment 12, wherein the cancer expresses caveolin 1 (Cav-1).

Embodiment 14. The method of embodiment 12 or embodiment 13, wherein the cancer expresses an increased amount of Cav-1 compared to a control, wherein the control comprises a non-cancerous cell of a same tissue type as the cancer.

Embodiment 15. A method of treating a cancer in a subject in need thereof comprising:
a) determining whether the cancer expresses caveolin 1 (Cav-1); and
b) if the cancer is determined to express Cav-1 in a), administering a therapeutically effective amount of an albumin conjugate of any one of embodiments 1-10 or a pharmaceutical composition of embodiment 11.

Embodiment 16. The method of embodiment 15, wherein determining whether the cancer expresses Cav-1 comprises determining a level of Cav-1 in a sample from the cancer.

Embodiment 17. The method of embodiment 16, wherein determining the level of Cav-1 comprises determining a level of Cav-1 polypeptide expression or functionality in the sample.

Embodiment 18. The method of embodiment 16, wherein determining the level of Cav-1 comprises determining a level of Cav-1 polynucleotide expression or functionality in the sample.

Embodiment 19. A method of treating a cancer in a subject in need thereof comprising:
a) determining whether the cancer expresses an increased amount of caveolin 1 (Cav-1) compared to a control, wherein the control comprises a non-cancerous cell of a same tissue type as the cancer; and
b) if the cancer is determined to express an increased amount of Cav-1 in a), administering a therapeutically effective amount of an albumin conjugate of any one of embodiments 1-10 or a pharmaceutical composition of embodiment 11.

Embodiment 20. The method of embodiment 19, wherein determining whether the cancer expresses an increased amount of Cav-1 compared to the control comprises comparing a level of Cav-1 in a sample from the cancer to a level of Cav-1 in a sample from the control.

Embodiment 21. The method of embodiment 19, wherein comparing a level of Cav-1 comprises comparing a level of Cav-1 polypeptide expression or functionality in the sample from the cancer to a level of Cav-1 polypeptide expression or functionality in the sample from the control.

Embodiment 22. The method of embodiment 19, wherein comparing a level of Cav-1 comprises comparing a level of Cav-1 polynucleotide expression or functionality in the sample from the cancer to a level of Cav-1 polynucleotide expression or functionality in the sample from the control.

Embodiment 23. The method of any one of embodiments 12-22, wherein the cancer is selected from melanoma, mesothelioma, a sarcoma, pancreatic, lung, breast, head and neck, urothelial, bladder, kidney, esophageal, and prostate cancer.

Embodiment 24. The method of any one of claims 12-23, wherein the albumin conjugate or pharmaceutical composition are administered in combination or alternation with one or more additional anticancer agents.

Embodiment 25. An albumin conjugate of any one of embodiments 1-10 or a pharmaceutical composition of claim 11 for use in treating a cancer in a subject in need thereof.

Embodiment 26. The albumin conjugate of embodiment 25, wherein the cancer expresses caveolin 1 (Cav-1).

Embodiment 27. The albumin of embodiment 25 or embodiment 26, wherein the cancer expresses an increased amount of Cav-1 compared to a control, wherein the control comprises a non-cancerous cell of a same tissue type as the cancer.

Embodiment 28. The albumin conjugate of any one of embodiments 25-27, wherein the cancer is selected from melanoma, mesothelioma, a sarcoma, pancreatic, lung, breast, head and neck, urothelial, bladder, kidney, esophageal, and prostate cancer.

Embodiment 29. Use of an albumin conjugate of any one of embodiments 1-10 in the manufacture of a medicament for treating a cancer in a subject in need thereof.

Embodiment 30. The albumin conjugate of embodiment 29, wherein the cancer expresses caveolin 1 (Cav-1).

Embodiment 31. The albumin of embodiment 29 or embodiment 30, wherein the cancer expresses an increased amount of Cav-1 compared to a control, wherein the control comprises a non-cancerous cell of a same tissue type as the cancer.

Embodiment 32. The albumin conjugate of any one of embodiments 29-31, wherein the cancer is selected from melanoma, mesothelioma, a sarcoma, pancreatic, lung, breast, head and neck, urothelial, bladder, kidney, esophageal, and prostate cancer.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

To further illustrate the principles of the present disclosure, the following examples are put forth so as to provide those of ordinary skill in the art with a complete disclosure and description of how the compositions and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. SSH20 Demonstrates Significant Efficacy in Caveolin-1 Expressing Tumors

In recent years, human serum albumin (HSA) has been characterized as an ideal drug carrier in the cancer arena. Caveolin-1 (Cav-1) has been established as the principal structural protein of caveolae and thus, critical for caveolae-mediated endocytosis. Cav-1 has been shown to be overexpressed in cancers of the lung and pancreas, amongst others. We found that Cav-1 expression plays a critical role in both HSA uptake and response to albumin-based chemotherapies. As such, developing a novel albumin-based chemotherapy that is more selective for tumors with high Cav-1 expression or high levels of caveolar-endocytosis could have significant implications in biomarker-directed therapy. Herein, we present the development of a novel and effective HSA-SN-38 conjugate (SSH20). We find that SSH20 uptake decreases significantly by immunofluorescence assays and western blotting after silencing of Cav-1 expression through RNA interference. Decreased drug sensitivity occurs in Cav-1 depleted cells using cytotoxicity assays. Importantly, we find significantly reduced sensitivity to SSH20 in Cav-1-silenced tumors compared to Cav-1-expressing tumors in vivo. Notably, we show that SSH20 is significantly more potent than irinotecan in vitro and in vivo. Together, we have developed a novel HSA-conjugated chemotherapy that is potent, effective, safe, and demonstrates improved efficacy in high Cav-1 expressing tumors.

Here, after testing over 30 different formulations of HSA to cytotoxic chemotherapies, we have identified a compound with high potency. This novel compound, in which we covalently conjugated HSA to SN-38, is designated SSH20. We show that SSH20 is robustly stable and soluble in aqueous solvents, while maintaining high potency relative to irinotecan. SSH20 demonstrates significant efficacy in both in vitro and in vivo pancreatic and lung cancer models. Furthermore, our results reveal that SSH20 targets cancer cells that express high levels of Cav-1, potentially providing a predictive biomarker to determine which patients would benefit most from SSH20.

### Materials and Methods

### Treatments, Reagents, Antibodies

Human serum albumin (HSA) solution (25%) was purchased from Octapharma (Paramus, NJ). SN38 (7-ethyl-10-hydroxy-camptothecin) was purchased from Ark Pharm, Inc. (Arlington Heights, IL). Succinic anhydride, N,N′-dicyclohexylcarbodiimide (DCC), N-hydroxysuccinimide (NHS), and 1,8-diazabicyclo [5.4.0]undec-7-one (DBU) were purchased from Sigma-Aldrich Chemical Co. (St. Louis, MO). Solvents, buffers, and salts, including dimethylformamide (DMF), acetone, chloroform, sodium carbonate, HEPES, and PBS, were purchased from Fisher Scientific (Hampton, NH). Irinotecan (West-Ward Pharmaceuticals, Eatontown, NJ) and SSH20 were dissolved in 0.9% saline. SN-38 was dissolved in DMSO. RPMI 1640 media, Minimum Essential Media (MEM), penicillin (100 U/ml)-streptomycin (100 μg/ml), and 0.25% w/v trypsin/1 mM EDTA were purchased from Gibco Life Technologies (Grand Island, NY). Dulbecco's Modified Eagle Medium (DMEM) and phosphate-buffered saline (PBS) were purchased from GE Healthcare BioSciences (Pittsburgh, PA). Fetal Bovine Serum (FBS) and lyophilized powder Human Serum Albumin (HAS) was purchased from Millipore-Sigma (St. Louis, MO). Cleaved caspase 3, cleaved PARP, human albumin, and GAPDH primary antibodies were purchased from Cell Signaling Technology (Danvers, MA). Caveolin-1 primary antibody (N-20) was purchased from Santa Cruz Biotechnology (Santa Cruz, CA). Anti-rabbit immunofluorescent secondary antibodies were purchased from LI-COR Biosciences (Lincoln, NE). Alexa Fluor Secondary Antibodies were purchased from Invitrogen (Waltham, MA).

### SSH20 Synthesis and Purification

SN38 and succinic anhydride were dissolved in 6 mL DMF at a molar ratio of 1:1.1. Equimolar ratio of DBU to SN38 was then added to the solution. The mixture was covered by aluminum foil and stirred overnight at room temperature to obtain SN38-suc. Next, 1.1× of DCC and NHS were added to the same mixture and stirred at room temperature for 6 hours to obtain SN38-suc-NHS. For HSA conjugation, a 25% HSA solution was diluted in 50 mM HEPES buffer to 10% and pH was titrated to pH 8.5 using 1M sodium carbonate. Then, 20× of the above-synthesized SN38-suc-NHS was added to the HSA/HEPES solution dropwise and the mixture was stirred at room for 4 hours to obtain SSH20. The solution pH was monitored and maintained at ˜pH 8.5 using 1M sodium carbonate. After HAS conjugation, the isourea (DCU) reaction byproduct was removed by centrifugation at 10,000×g for 15 minutes. The supernatant was collected in polypropylene conical tubes with 10 mL per tube. 40 mL of pre-chilled acetone (at −20° C.) was added to each tube to form SSH20 precipitate. The protein/acetone mixture was incubated at −80° C. for one hour and SSH20 precipitate was spun down at 4000×g for 20 minutes at 4° C. The supernatant was removed, and the protein pellet was dried in a desiccator under vacuum for 10 minutes to remove residual acetone. The pellet was resuspended in PBS to obtain a crude SSH20 solution. The crude SSH20 was dialyzed against PBS overnight using 10 kDa dialysis cassettes to remove residual acetone, SN38, and byproducts. The purified SSH20 was sterile-filtered through 0.45 μm PES membrane to remove invisible aggregates. The final product was stored at −20° C.

### SSH20 Characterization

For MALDI-TOF mass spectrometry, SSH20 and HSA were desalted using a PD-10 column (GE Healthcare Life Science) and buffer-exchanged to deionized water. The protein concentrations were measured by BCA assay from Thermo Scientific (Waltham, MA) and diluted to 1 mg/mL using deionized water. The protein samples were then analyzed on a Bruker ultrafleXtreme MALDI-TOF-TOF MS. For SDS-PAGE characterization, SSH20 was diluted to 2 mg/mL using PBS. HAS standard was prepared in PBS at 2 mg/mL (calibrated by NanoDrop OD280 using its extinction coefficient of 35,700 M⁻¹ cm⁻¹). Sample buffer (BioRad, Hercules, CA) with 5% β-mercaptoethanol (β-ME) was added to the protein samples and the mixtures were incubated at 100° C. for 5 minutes. The denatured protein samples were then run on a 4-15% TGX precast protein gel (BioRad) along with Precision Plus Protein dual-color standard (BioRad) at a constant voltage of 100V for 75 minutes. The gel was fixed with 40% ethanol/10% acetic acid for 15 minutes, rinsed with deionized water, and stained with QC Colloidal Coomassie Stain (BioRad) overnight. The stained gel was destained with deionized water and imaged on a ProteinSimple Fluorochem M Imager. For organic solvent extraction, 100 μL chloroform was added to an equal volume of SSH20 and mixed on a high-speed vortex for one minute. The chloroform layer was separated by centrifugation and analyzed by HPLC. The analysis was run with an isocratic 40% acetonitrile/60% water mobile phase and on a C18 column (Kromasil 100-5-19, 4.6×150 mm). The PDA was set at 365 for detection.

### Cell Culture

MIA-PaCa-2 and H23 cells were authenticated (via short tandem repeat profiling). Stable shRNA Cav-1 knockdown MIA-PaCa-2 and H23 cells were generated as described previously (11). Cells were maintained at 37° C. in 5% CO₂ in DMEM (MIA-PaCa-2) or RPMA 1640 (H23) media supplemented with 10% FBS and 1% penicillin/streptomycin. Cells were cultured for no more than 3 months continuously before rethawing cells from an early passage. Cells were routinely tested for Mycoplasma. SN-38, SSH20, irinotecan, and methyl-beta-cyclodextrin were added to media with a final vehicle concentration of no more than 0.1/%.

### Cellular Proliferation Assay

AlamarBlue proliferation assay was performed according to the manufacturer's instructions (BioRad Antibodies, Oxford, UK). Briefly, cells were seeded in 96-well plates in 4 replicates at a density of 100-300 cells per well in 100 μL medium and treated as described. Seventy-two hours after plating, alamarBlue reagent was added and incubated at 37° C. for 4-8 hours, and absorbance was measured at 570 and 600 nm.

### Immunofluorescence

Cells were plated on coverslips and treated with or without FITC-HAS, SN-38, or SSH20. Cells were pulsed either 1 hour with 0.1% FITC-HAS or 2 hours with 250 nM SN/38/SSH20 prior to fixation. When applicable, in order to remove membrane-bound albumin, two acid/salt washes were performed with 0.1M glycine and 0.1M NaCl, pH 3.02 for 2 minutes each, followed by two washes with phosphate buffer saline (PBS). Cells were then fixed with 2% paraformaldehyde for 15 minutes at room temperature and washed 1×PBS 2 times for 5 minutes each. Cells were incubated with 0.1% Triton-x-100 for 10 min on ice to permeabilize, then washed twice with 1×PBS prior to blocking with 3% bovine serum albumin in 1×PBS overnight at 4° C. In a humidified chamber, primary antibodies (1:50) in blocking buffer were added and incubated for 1 hour at 4° C., followed by three 10 minute rinses with blocking buffer. Secondary antibody (conjugated to Alexa Fluor 488, 1:1000) was added along with DAPI for 1 hour at room temperature. Cells were then rinsed, and coverslips were mounted onto slides and then sealed. Cells were then imaged with a confocal microscope with locked settings across the control and respective experimental conditions. For auto-fluorescence experiments with SN-30 and SSH20, images were taken at an emission wavelength of 543 nm. ImageJ was used to measure the fluorescence intensity of individual cells.

### Immunoblotting

For assessment of Cav-1 expression, octyl-β-D-glucopyranoside (Millipore-Sigma; St. Louis, MO) was added at 60 mM final concentration to RIPA buffer (Thermo Fisher Scientific; Waltham, MA) containing protease and phosphatase inhibitor cocktails (Roche; Basel, Switzerland). Protein concentration was determined with a Dc Protein Assay Kit (BioRad; Hercules, CA). For albumin immunoblots, 2 acid/salt washes with 0.1M glycine and 0.1 M NaCl, pH 3.02 were performed on ice for 2 min each, followed by a PBS wash prior to cell lysis. Proteins were resolved bd SDS/PAGE and transferred to nitrocellulose membranes. Membranes were incubated in 5% BSA in TBS-Tween blocking buffer for 1 hour at room temperature. Primary antibodies were allowed to bind overnight at 4° C., and used at a dilution of 1:1,000. After washing in TBS-Tween three time for 10 minutes each, the membranes were incubated with immunofluorescent secondary antibodies at a 1:5,000 dilution for 1 hour at room temperature. Membranes were washed with TBS-Tween prior to imaging via LI-COR Odyssey CLx Imaging System (Lincoln, NE).

### In Vitro Serum Stability

Briefly, 180 μL of SSH20 was added to 1620 μL of pooled human serum off the clot (Innovative Research, Novi, MI) to form a 10% SSH20 solution, which was incubated at 37° C. Aliquots were collected at preset time points, a fixed amount of internal standard (10-hydroxycamptothecin, 10-HCPT) was added to each sample aliquot and the solutions were mixed thoroughly on vortex prior to protein precipitation by acetonitrile. Protein precipitates were pulled down by 17,000×g centrifugation for 5 minutes. The dissociated SN-38 in the supernatant was analyzed by reverse-phase HPLC, using parameters described above, detected by PDA at 368 nm. SN-38/internal standard peak ratios were correlated with an SN-38 standard curve (using the same preparation method) to obtain the concentrations of released SN-38. The plot of the kinetics of SN-38 release was generated by Prism, and the half-life was calculated by one-phase exponential association.
In Vivo Studies Eight- to ten-week-old male athymic nude mice (Taconic Farms Inc.) were caged in groups of five or less and fed a diet of animal chow and waster ad libitum. MIA-PaCa-2 or H23 cells (2×10⁶) with stable control shRNA (shCtrl) or shCav-1 were injected subcutaneously into the flanks of athymic nude mice. Treatment regimens were started once tumors reached approximately 100-200 mm³ in size (typically 1-3 weeks post-injection). SSH20 (10 mg/kg) and the molar equivalent of irinotecan in 0.9% saline was administered intravenously via retro-orbital injection accordingly. To obtain a tumor growth curve, perpendicular diameter measurements of each tumor were measured every 1-5 days from the first day of injection with digital calipers, and volumes were calculated using the formula (L×W×W)/2. Weight and clinical signs of toxicity were monitored several times per week.

### Statistical Analysis

Data are presented as the mean f standard error of the mean (SEM) for proliferation assays, immunofluorescence intensity, and tumor growth experiments. The group comparisons of the percent change in tumor volume were performed at individual time points. Statistical comparisons were made between the control and experimental conditions used the unpaired two-tailed Student t-test with significance assessed at P<0.05. Log-rank (Mantel-Cox) test was performed for comparison of survival (Kaplan-Meier) curves between control and SSH20 treatment groups with significance assessed at P<0.05. GraphPad Prism (GraphPad Software Inc.) was used to perform the statistical analyses.

### Results

### SSH20 Showed Best Activity of SN-38 Conjugates Explored

The following conjugates between SN-38 and HSA were prepared and analyzed for their inhibitory activity against the MP2 cell line:

| Conjugate Name | Crosslinking Agents | IC₅₀ (μM) |
|---|---|---|
| SN-38 (control) | — | 0.495 |
| SSH20 | Succinic anhydride | 0.0622 |
| SCH10 | Carbonyldiimidazole | >1 |
| SCH20 | Carbonyldiimidazole | >>1 |
| SDSH10 | 1,8-diazabicyclo(5.4.0)undec-7- | 0.476 |
| | ene, succinic anhydride, 1-ethyl-3- | |
| | N,N- | |
| | dimethylaminopropylcarbodiimide | |
| | hydrochloride, N- | |
| | hydroxysuccinimide | |
| SDSH20 | 1,8-diazabicyclo(5.4.0)undec-7- | 0.6658 |
| | ene, succinic anhydride, 1-ethyl-3- | |
| | N,N- | |
| | dimethylaminopropylcarbodiimide | |
| | hydrochloride, N- | |
| | hydroxysuccinimide | |
| SMTH10 | 4-maleimidobenzoic acid chloride, | Cannot be obtained |
| | 2-iminothiolane | |

As can be seen, S12 had the greatest inhibitory effect of the conjugates tested. Surprisingly, a majority of the other conjugates tested were found to have worse inhibitory activity against the MP2 cell line compared to unconjugated SN-38, with only SSH20 showing a sizeable improvement compared to the unconjugated compound.

### Characterization of SSH20

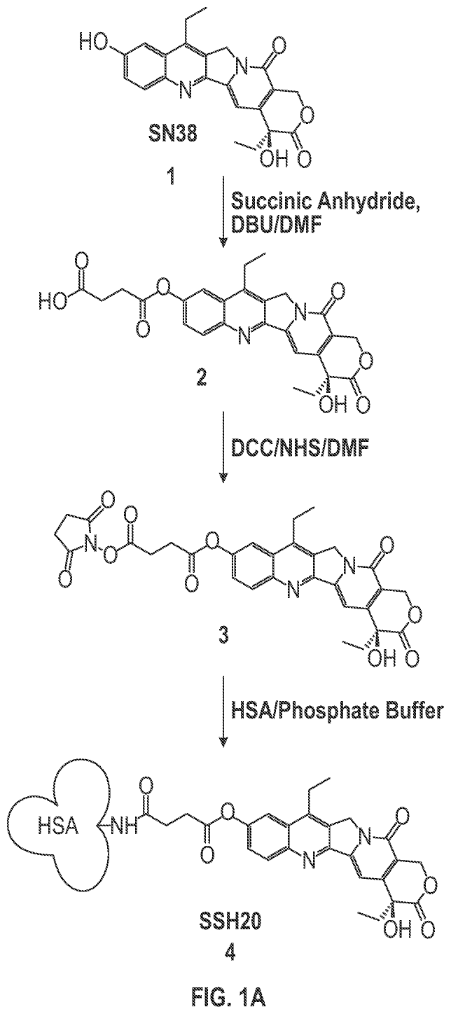

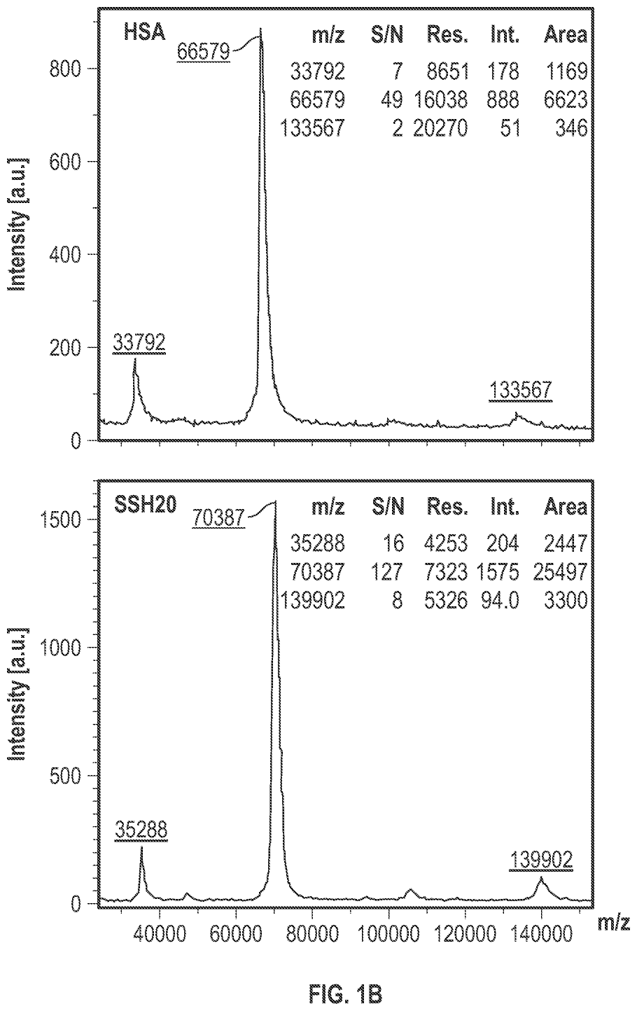

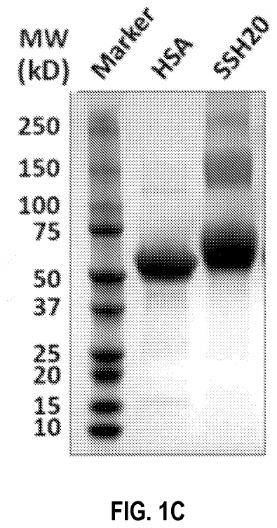

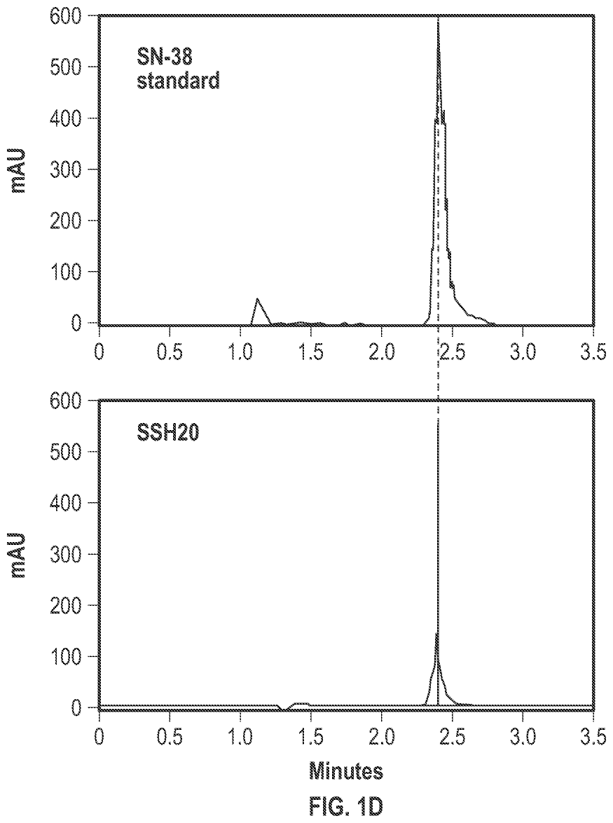

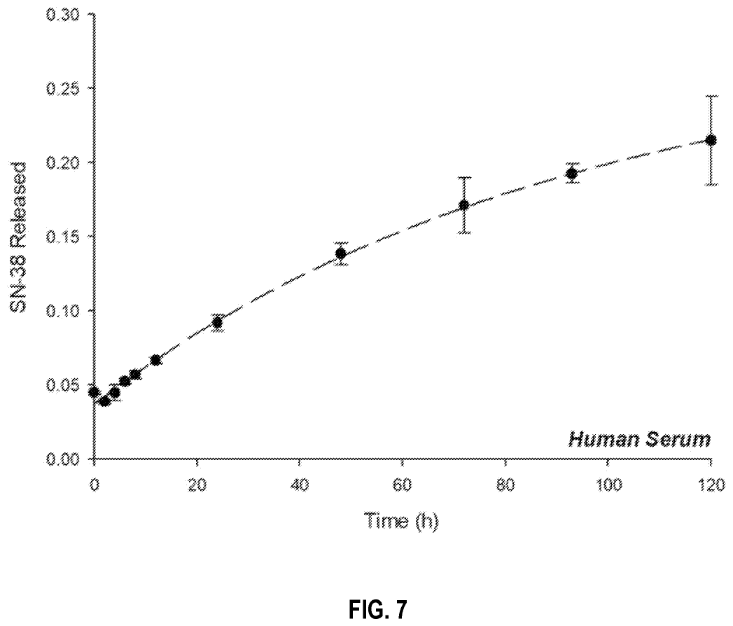

The reaction scheme for the synthesis of SSH20 is shown in FIG. 1A. The SN38 and HSA concentration were determined by UV absorption at 360 nm and BCA protein assay (Thermo Scientific, Waltham, MA), respectively. The results showed that SSH20 conjugate contained an average of 6.9 SN38s per HSA molecule. Similar results were also obtained by MALDI-TOF mass spectrometry (FIG. 1B), which indicated an average of 7.75 SN38s per HAS molecule. To examine the linkage between SN38 and HSA, SDS-PAGE (FIG. 1C) and organic solvent extraction (FIG. 1D) were performed to analyze HSA and SN38, respectively. As shown in FIG. 1C, the major band of SSH20 shifted upwards compared to that of HSA, suggesting that SN38 molecules were covalently bound to HSA, resulting in higher molecular weight. Similar results were obtained by organic solvent extraction. The extraction percentage of SN38 from SSH20 was <1%, normalized to an equal concentration of standard (FIG. 1D), suggesting that SN38 molecules were bound to HSA through covalent ester bonds and could not be extracted out into an organic solvent. To determine the drug-releasing effect of plasma esterase on SSH20, SSH20 serum stability was analyzed. The results indicated the release of SN-38 from SSH20 occurred in a time-dependent manner with a half-life of 64.2 hours (FIG. 7).

### Cav-1 Expression Mediates Uptake of HSA In Vitro

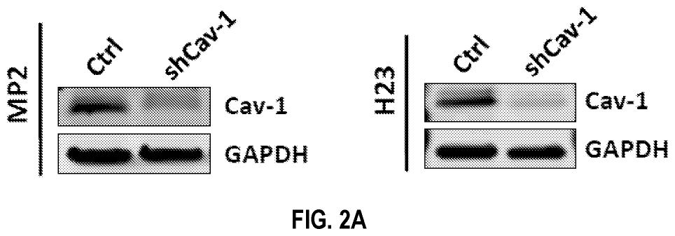

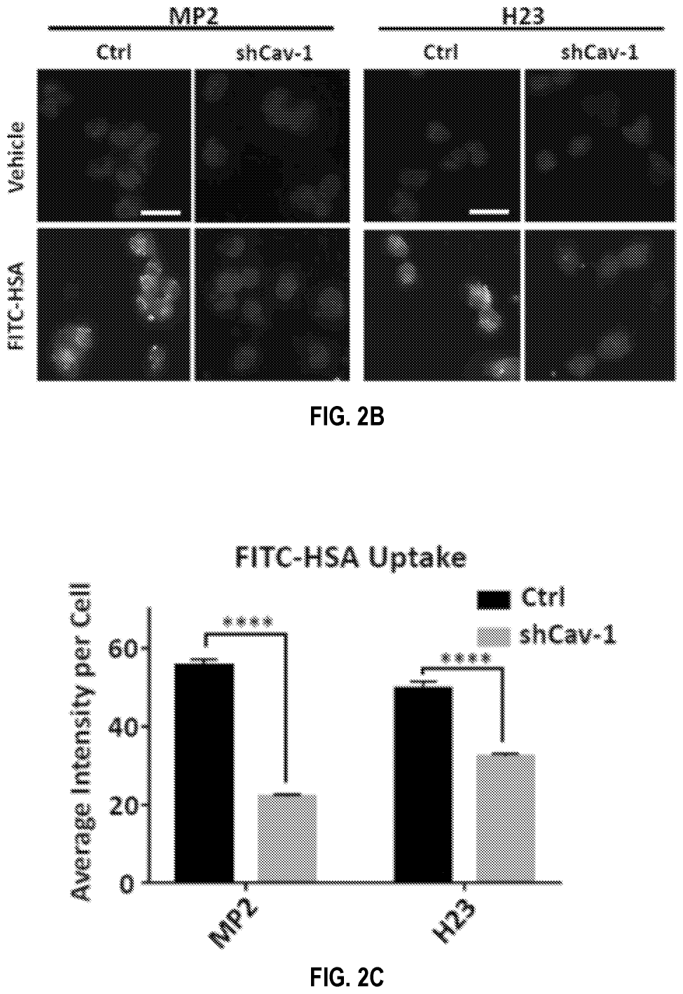

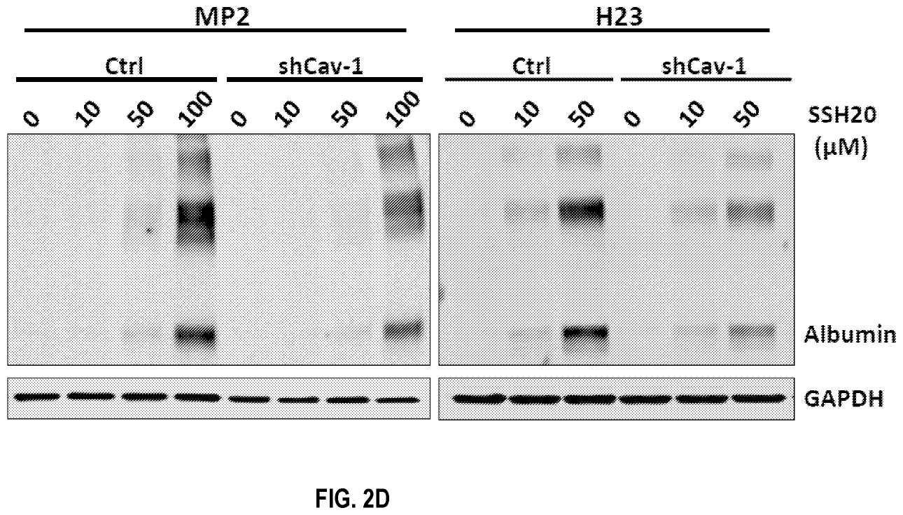

In order to first verify the role of Cav-1 expression in albumin uptake into cells, we compared uptake between our stable shCtrl and shCav-1 in MIA-PaCa-2 (MP2) and H23 cell lines (previously generated (11)). We first confirmed that Cav-1 expression was depleted in our shCav-1 cells relative to shCtrl cells via immunoblotting (FIG. 2A). Then, we pulsed cells with FITC-HSA (0.01%) for 1 h in regular growth medium to compare uptake of albumin in shCav-1 versus shCtrl cells by direct immunofluorescence (FIG. 2B). We then measured FITC channel fluorescence intensity of individual cells and observed significant reduction of albumin uptake in shcav-1 cells in both MP2 and H23 groups (FIG. 2C). As an initial test of whether this pattern of HSA uptake was maintained after covalent conjugation to SN-38, we performed immunoblotting for human-specific albumin after cells were pulsed for 1 h with increasing doses of SSH20. Albumin levels were to be higher in shCtrl than shCav-1 cells (FIG. 2D). These results support our previous finding that cancer cells with higher expression of Cav-1 are able to internalize albumin more abundantly than low Cav-1 expressing cancer cells. Furthermore, the properties of HSA resulting in uptake through caveolae-mediated endocytosis are maintained in our SN-38 conjugate, SSH20.

SSH20 Targets Cancer Cell with High Cav-J Expression In Vitro

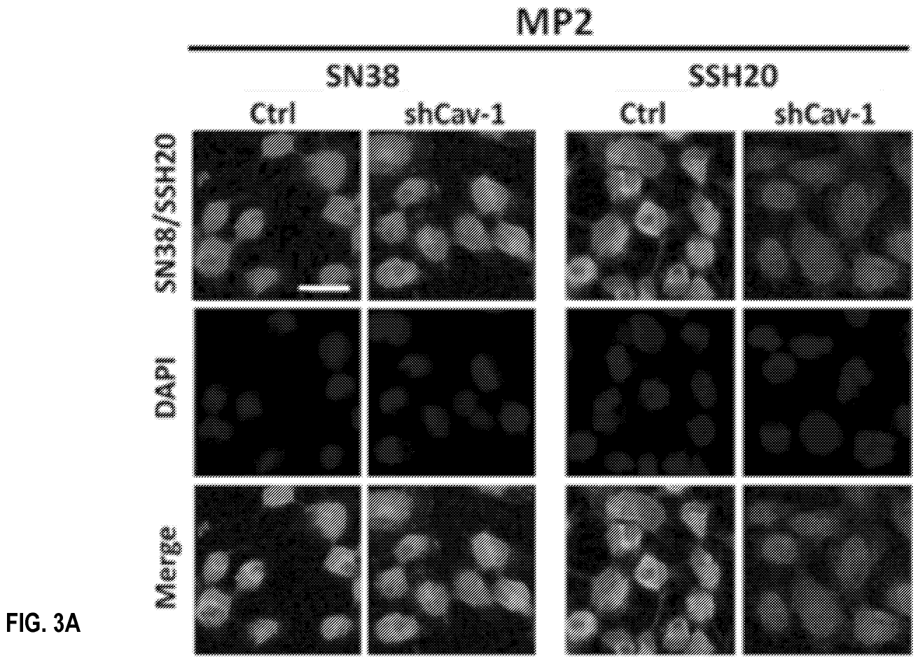

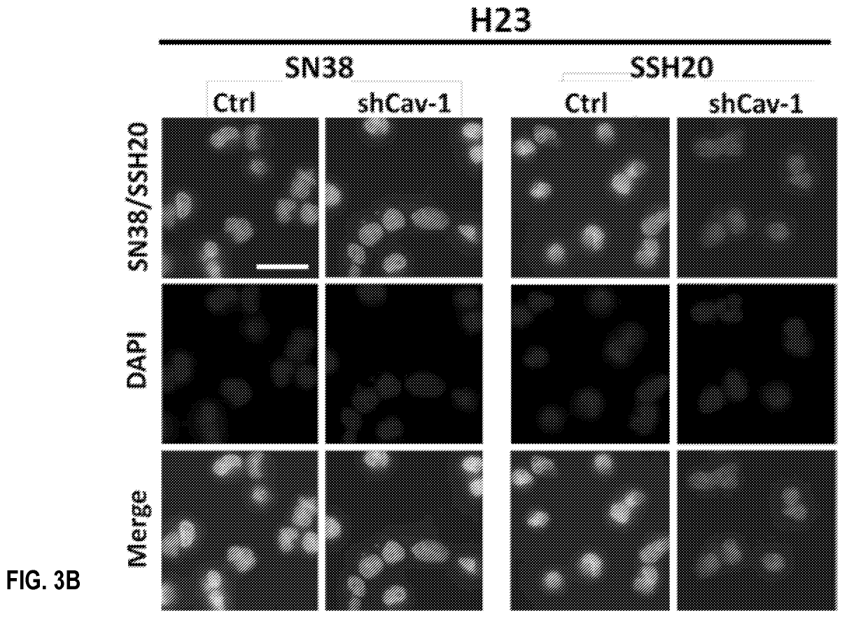

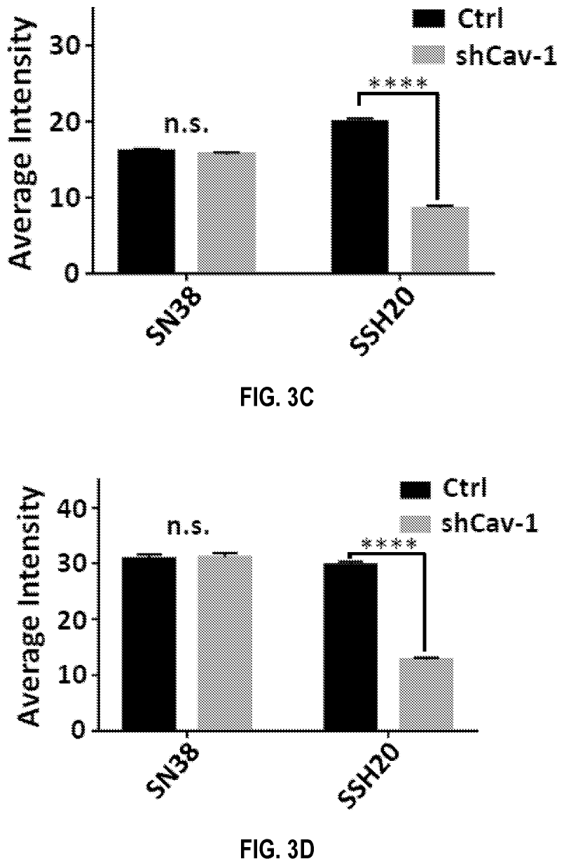

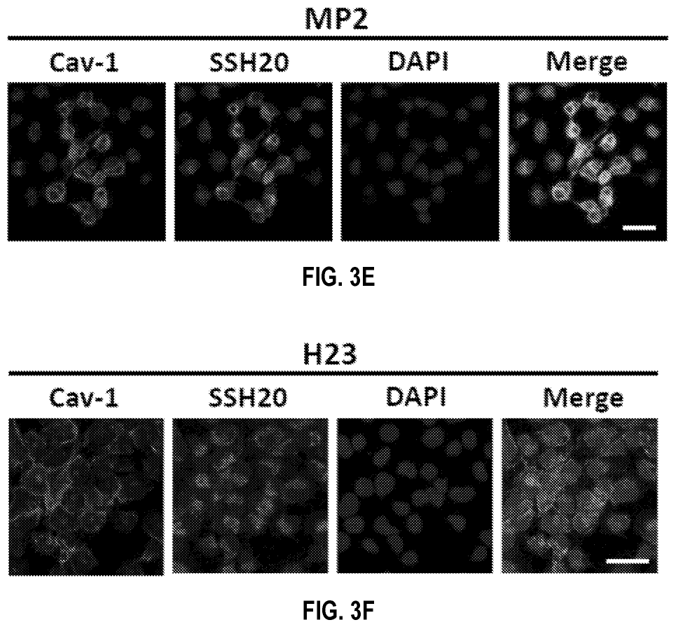

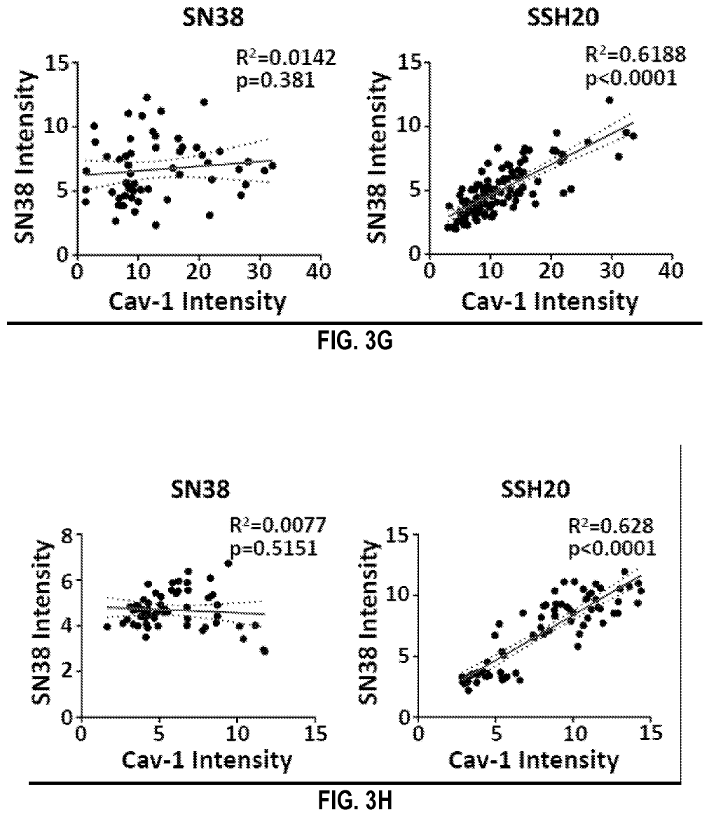

Next, we tested the ability of SSH20 to target cells with high (shCtrl) versus low Cav-1 (shCav-1) expression in our isogenic cell lines. Taking advantage of the autofluorescent property of SN-38, we pulsed cells with SN-38 or SSH20 for 2 h and determined the amount of drug that was internalized by direct fluorescence microscopy. Upon imaging, we observed a marked difference in SN-38 by direct fluorescence microscopy. Upon imaging, we observed a marked difference in SN-38 autofluorescent signal between shCtrl and shCav-1 SSH20-treated cells, but no discernable difference in SN-38-treated cells (FIG. 3A-B). This was confirmed quantitatively by comparative analysis of the fluorescence intensity. The intensity measured in the shCav-1 cells was significantly less than that in the shCtrl cells in both MP2 and H23 isogenic cell lines (FIG. 3C-D). Furthermore, we co-cultured shCtrl and shCav-1 cells at a 1:1 ratio and, as before, pulsed the cells with SN-38 or SSH20 for 2 h. We performed direct fluorescence for SN-38 and indirect immunofluorescence for Cav-1 protein with a secondary-antibody conjugated with AlexaFluor-568. After acquiring images (FIG. 3E-F), we measured the unicellular fluorescent intensity of both the SN-38 autofluorescent signal (green) and Cav-1-AF568 (red) channels, and generated a scatterplot using these values (FIG. 3G-H). In both MIA-PaCa-2 (FIG. 3E, 3G) and H23 (FIG. 3F, 3H) co-culture experiments, Pearson correlation coefficient analysis revealed a highly significant correlation between Cav-1 expression and SSH20, but not SN38. Taken together, these data strongly suggest that cancer cells with higher Cav-1 expression uptake SSH20 more readily, and further support the hypothesis that SSH20 targets cancer cells with increased levels of caveolae-mediated endocytosis.

### Knocking Down Cav-1 Expression Reduces Sensitivity of SSH20 In Vitro

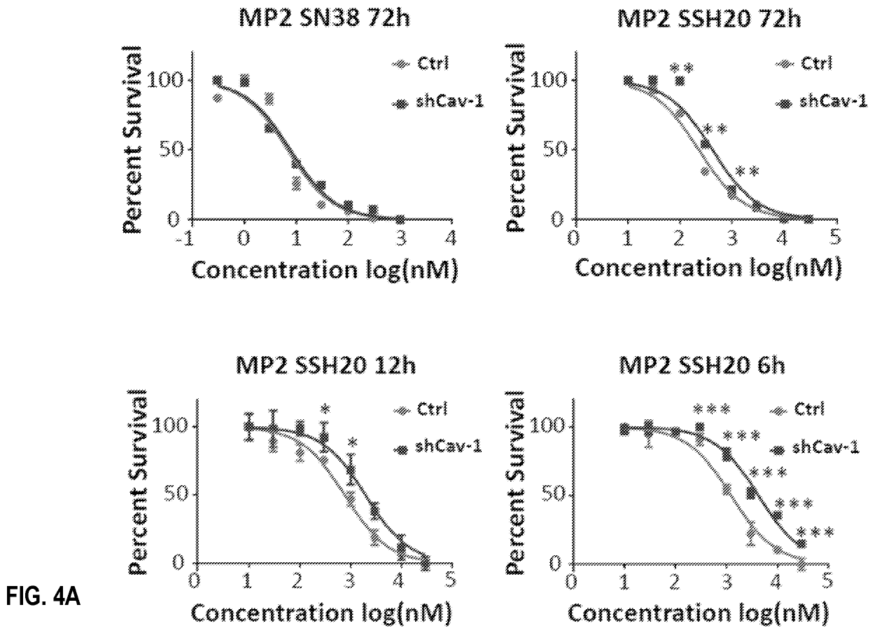

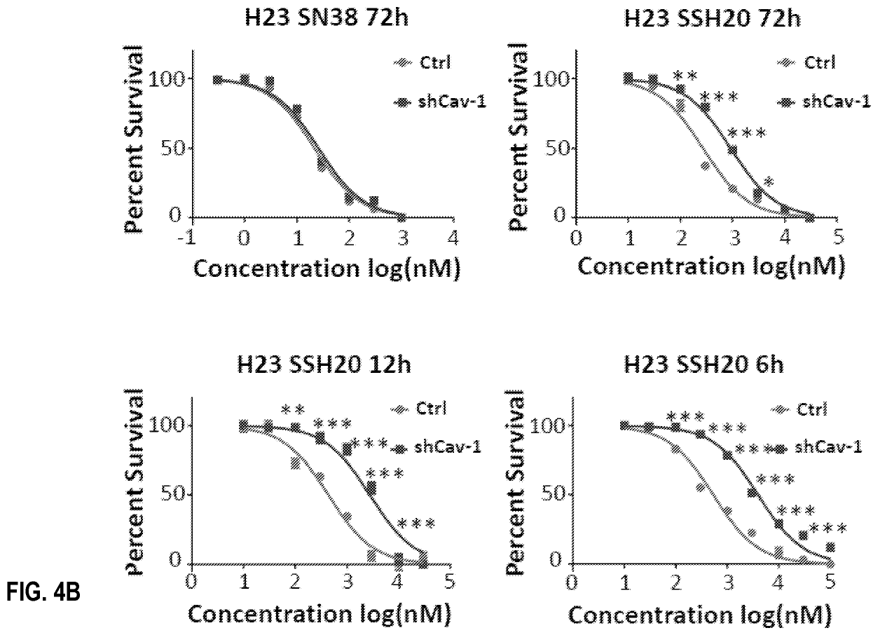

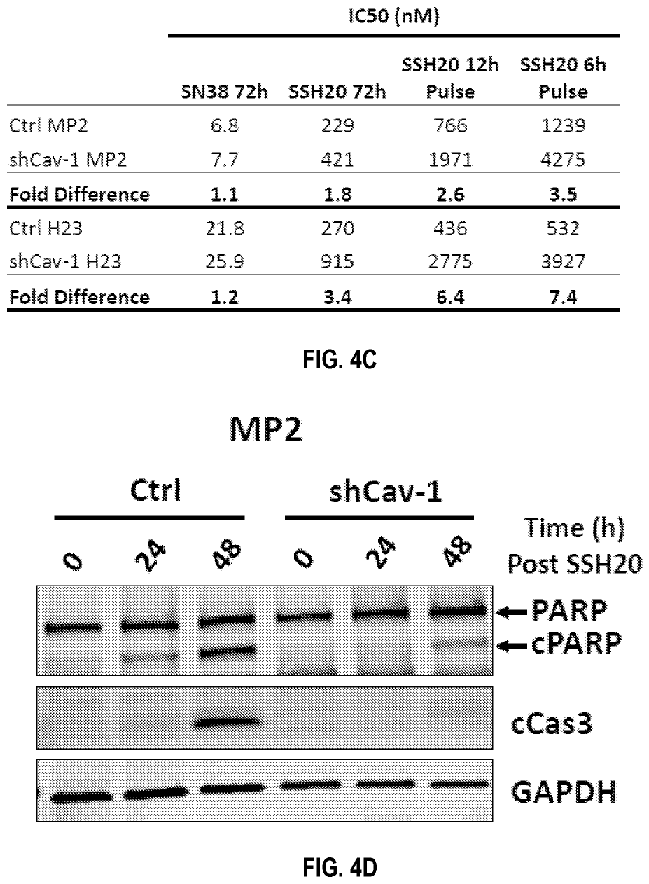

As differences in Cav-1 expression resulted in significantly different SSH20 uptake, we tested whether this would directly translate to a similar trend in sensitivity to SSH20. Using cytotoxicity assays, we found that Cav-1 depletion resulted in decreased sensitivity of the cells to SSH20, but not to SN-38 after 72 h treatment (FIG. 4A-B). In order to evaluate whether the observed difference in sensitivity was, at least in part, a result of the rate of drug internalization, we pulse-treated the cells for shorter time intervals of 6 and 12 h. An increased differential of SSH20 sensitivity between shCav-1 and shCtrl cells was observed as the treatment time decreased, as noted by separation of the curves (FIG. 4A-C). In addition, Cav-1 depletion markedly reduced SSH20-induced apoptosis in MP2 cells (FIG. 4D). Taken together, these results indicate that reduction of Cav-1 expression decreases sensitivity of cells to SSH20, at least in part through reduced induction of apoptotic cell death and support that caveolae-mediated endocytosis is a critical factor of SSH20 sensitivity.

SSH20 Demonstrates Higher Potency than Irinotecan and Efficacy in Cav-1 Expression Tumor Models In Vivo

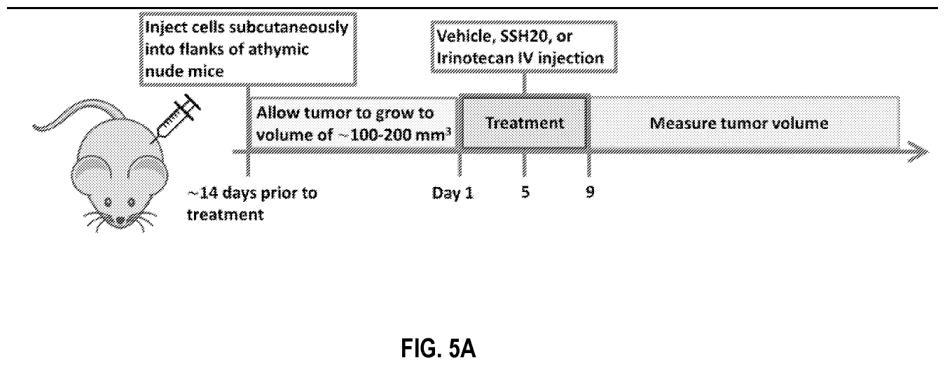

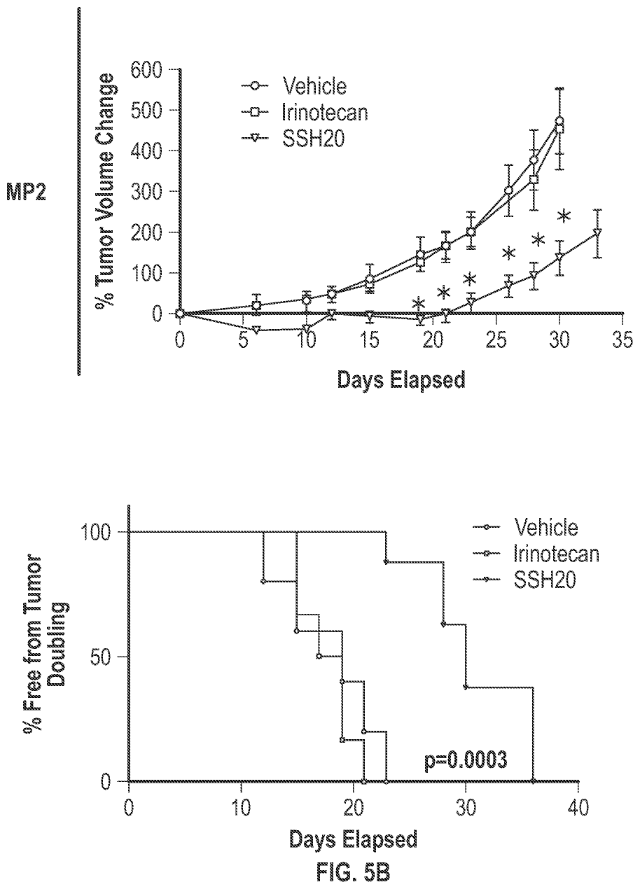

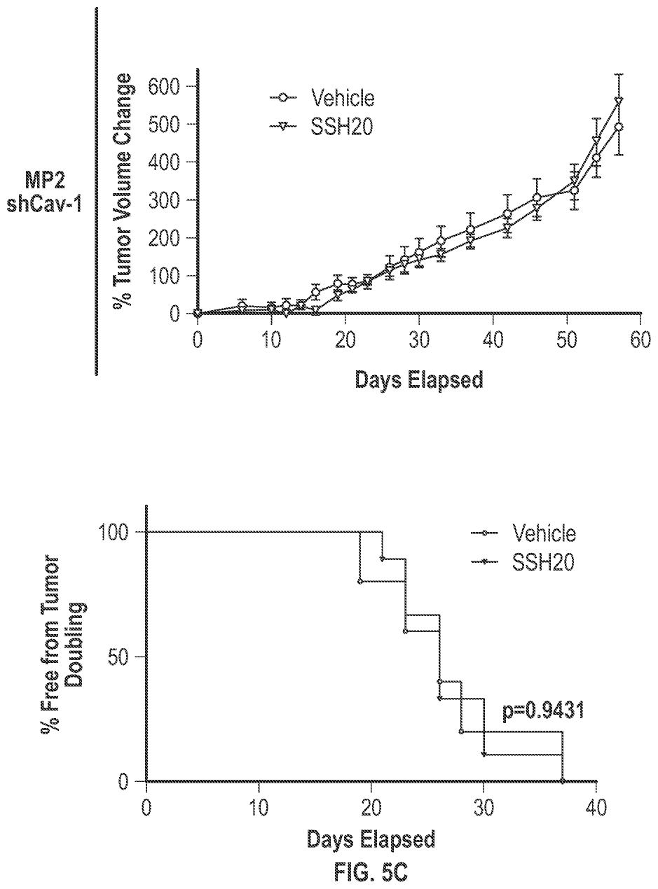

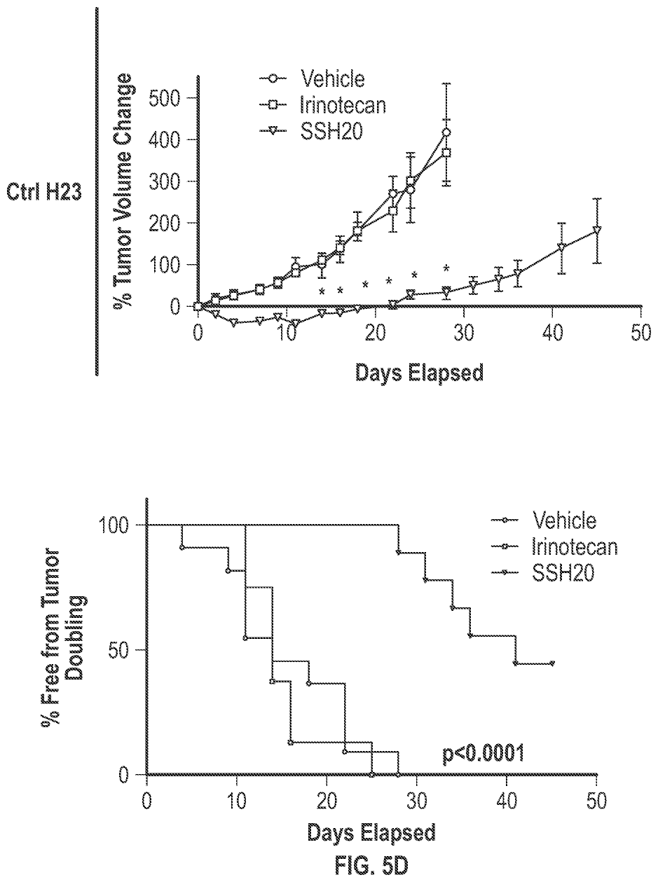

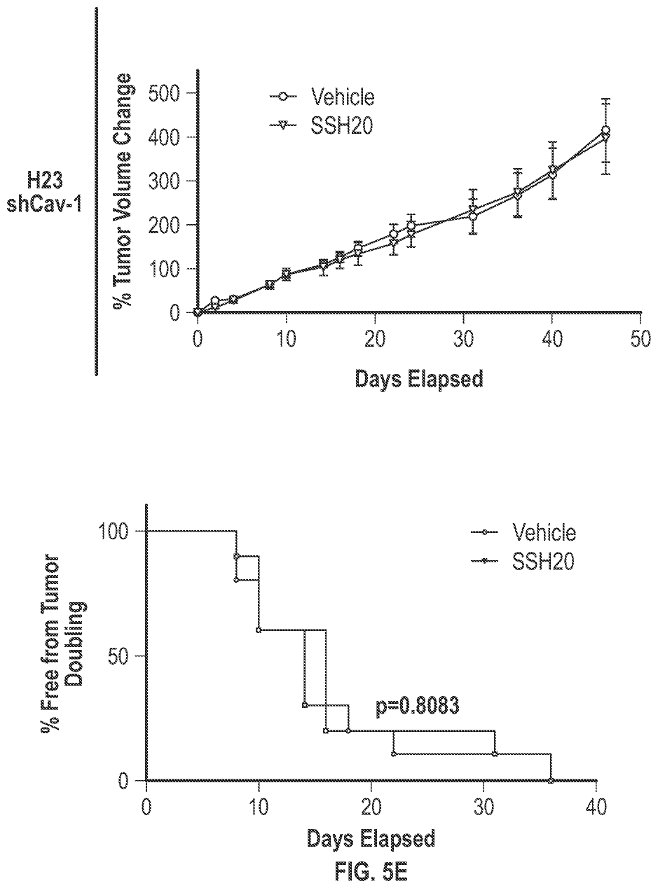

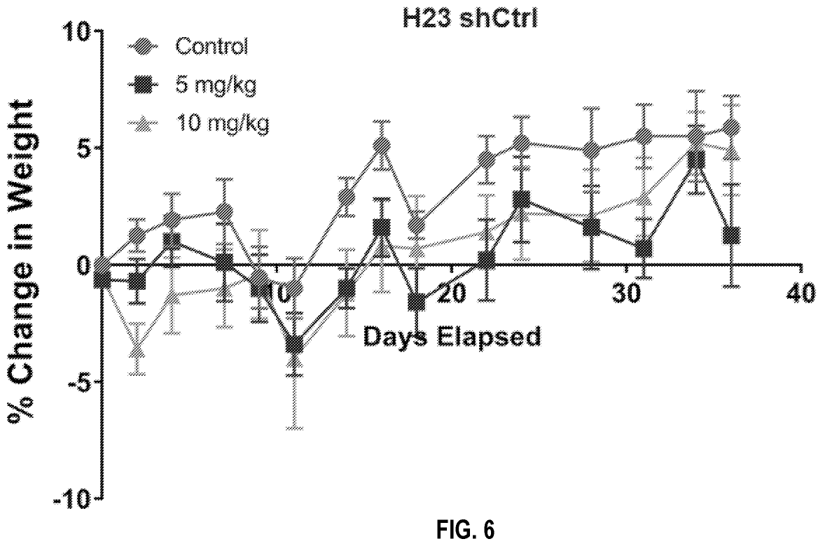

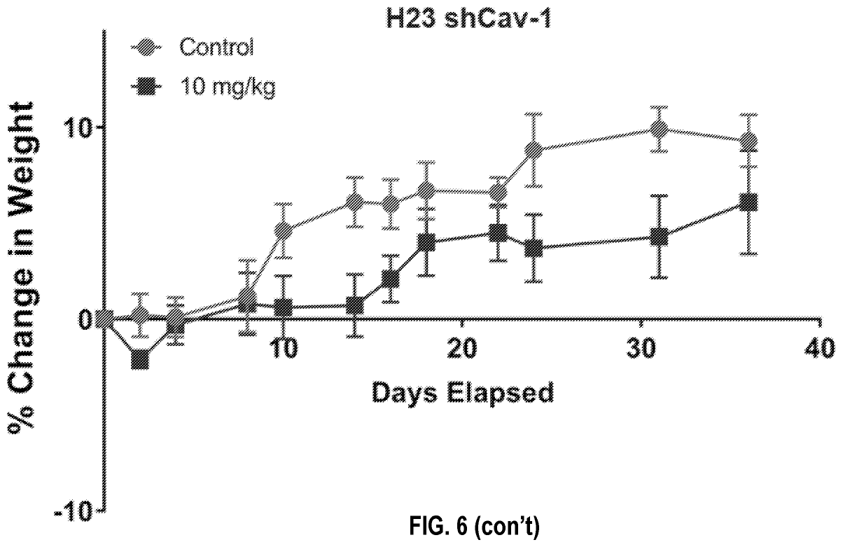

To explore the impact of Cav-1 expression on SSH20 sensitivity in vivo, we injected MP2-shCtrl/shCav-1 and H23-shCtrl/shCav-1 cells into the flanks of athymic nude mice, then treated the mice with or without SSH20 as indicated in FIG. 5A. SSH20 treatment significantly reduced tumor growth rate in shCtrl (Cav-1 proficient) groups (FIG. 5B, 5D left panels). However, no significant difference of tumor growth rate was found in shCav-1 xenografts with or without SSH20 treatment (FIG. 5C, 5E, left panels). In addition, tumor growth rate was not reduced by equivalent doses of irinotecan treatment in shCtrl-tumor bearing mice, given the lower potency of irinotecan at equivalent doses (FIGS. 5B and 5D, left panels). Kaplan-Meier survival curves based on percent of mice free from tumor doubling showed that SSH20 treatment markedly extended tumor doubling time in shCtrl groups, while no difference was detected in shCav-1 groups (FIG. 5B-E, right panels). Notable, mice tolerated treatment with SSH20 well, with no observable clinical signs of toxicity and minimal weight changes detected with doses of 5 or 10 mg/kg of SSH20.

### Discussion

Major issues related to success of novel therapeutics include improving drug delivery/solubility and predicting which tumors will respond best to the therapy. Irinotecan has long been used as a cancer therapeutic, and irinotecan is metabolized to the much more potent metabolite SN-38 which interacts with the nuclear enzyme topoisomerase I, resulting in irreversible double strand breaks and cell death (22,23). However, SN-38 cannot be solubilized into a form that is deliverable to humans previously. Here, we describe the development of a novel and stable HSA-conjugated SN-38 protein-drug nanoparticle conjugate termed SSH20 that demonstrates significant in vitro and in vivo efficacy in pancreatic and lung cancer models. Mice tolerate SSH20 treatment well with minimal signs of toxicity. Furthermore, SSH20 treatment demonstrates improved efficacy over irinotecan and is most effective in tumors with high Cav-1 expression.

As reported by previous studies, strategic conjugation with hydrophilic groups can solubilize SN-38 (24,25). Due to the covalent conjugation of SN-38 to HSA, SSH20 remains stable and soluble in water or saline solution. This allows for intravenous administration, providing a method of drug delivery not viable with free SN-38. SN-38 could be release from albumin conjugate to exhibit its activity through esterases, which are generally overexpressed by tumor cells, and our data indicated that SN-38 was slowly released during the 120-hr incubation (FIG. 7). Previous publications have indicated the advantage of HSA conjugation: biocompatible breakdown, increased pharmacokinetic half-life, and preferential tumor localization (5, 26, 27). Based on these, development of HSA drug conjugates has been considered an attractive approach to increase drug targeting. The success of nab-paclitaxel in the clinic is a prime example of an FDA-approved albumin-chemotherapeutic for the treatment of cancer (28,29). However, nab-paclitaxel is loosely bound to albumin by virtue of not being directly chemically conjugated, likely resulting in increased dissociation of paclitaxel from albumin before the drug reaches its intended tumor cell targets. Consequences of this are that more free paclitaxel would lead to increased normal tissue toxicity and less specificity for Cav-1 expressing tumor cells.

Numerous research groups have attempted to synthesize albumin-conjugated compounds (30-32). Interestingly, Sepheri et al. also developed an Sn-38-albumin conjugate and reported in vitro cytotoxicity and in vivo biodistribution and blood cytotoxicity in colon cancer (32). Our conjugate is distinct, as we conjugated the albumin to the phenol —OH of the SN-38 compound, while Sepheri et al. conjugated it to the aliphatic —OH. The study of SSH20 followed a screening of conjugates of SN-38 and HSA against the MP2 cell line for inhibitory activity, which unexpectedly revealed a wide range of IC₅₀ values for the different conjugates. The difference in —OH attachment between SSH20 and the conjugate from Sepheri et al. is enough to establish that SSH20 is a novel compound. In addition, SSH20 has a clear advantage as the conjugation synthesis is simpler without sacrificing purity. Furthermore, more molecules of SN-38 are conjugated to HSA in SSH20 compared to the Sepheri et al. strategy (˜7-8 vs ˜2-4). Finally, it achieves at least 99.99% purity which is significantly higher than the conjugate from Sepheri et al. Protein-drug conjugates must strike a balance between esterase-stability and drug releasability. Our studies support that esterases may function to degrade SSH20 and account for reduced efficacy in our models. Interestingly, mice have several fold higher plasma esterase activity than humans. Despite this, however, we still observed a dramatic effect in vivo, and we speculate that the therapeutic index could be widened in an esterase-depleted environment. In fact, this is one of the key issues in the design and development of antibody-drug conjugates, which typically have stayed away from highly stable linkers. The optimization of linker chemistry and characterization of ester linkage for SSH20 will be further established in our future studies.

It has been difficult to identify predictive biomarkers of irinotecan treatment effectiveness. In agreement with our previous work, we found that a further property attributed to used HSA as a drug carrier for SN-38 is potential Cav-1 mediated specificity. We have shown that both free HSA and SSH20 are internalized to a greater degree in cancer cells relative to the level of Cav-1 protein. Our results also indicate that increased uptake is accompanied by increased SSH20 sensitivity in lung and pancreatic cancer cells expressing high levels of Cav-1. Taken together, these results suggest that SSH20 likely has improved efficacy in high Cav-1 expressing tumors. This example provides preliminary support for the potential of SSH20 as a novel treatment approach against lung and pancreatic cancers with high Cav-1 expression. As Cav-1 is commonly overexpressed among these cancer types, using Cav-1 as a predictive biomarker would allow for improved likelihood of success in clinical trials and facilitate implementation of a more personalized approach for SSH20 treatment.

As we know, albumin is a nutrient source for tumor cells, is especially accumulated in the tumor microenvironment due to the EPR effect, which is likely an advantage of SSH20 as well. It is also important to note that we found SSH20 was significantly more potent than irinotecan in vivo (in Cav-1 expressing tumors). Importantly, there was no apparent different in toxicity between them in vivo. These findings, in addition to the tumor targeting properties resulting from the HSA group, provide evidence for SSH20 being a more effective alternative strategy for patients who would receive irinotecan according to the current standard of care.

Certainly, it is possible that tumor cells have additional uptake mechanisms of HSA-conjugated drugs aside from caveolae-mediated endocytosis. Indeed, in our in vitro studies, we found that the highest degree of separation between Cav-1 proficient (Ctrl) and Cav-1 deficient (shCav-1) cells in our IC50 experiments occurred with shorter periods of time of exposure of cells to SSH20 (i.e. 60 or 12 h versus 72 h). This suggests that SSH20 is preferentially taken up first by caveolae-mediated endocytosis but that other unidentified mechanisms of uptake may predominate at later time points. Nevertheless, the marked separation in efficacy in our in vivo experiments between Cav-1 proficient and Cav-1 deficient tumors clearly demonstrates that caveolae-mediated endocytosis is a profound mechanism of uptake for SSH20, and that Cav-1 expression is likely to be a very useful predictive biomarker. This degree of separation was not observed with nab-paclitaxel in our previous publication (11), which may be related to the instability and frequent dissociation of paclitaxel from albumin, and subsequent free diffusion of paclitaxel through the cell membrane (independent of Cav-1/caveolae-mediated endocytosis).

It is important to highlight the limitations of this example. One of the advantages of albumin bound drugs is based on the EPR effect, which we did not directly test in this example. Additionally, in our in vivo models, we have used subcutaneous flank mouse models for pancreatic cancer (and lung cancer), which do not recapitulate the stroma rick and native tumor microenvironment of pancreatic cancer. To better reflect that intrinsic TME, orthotopic implantation of human patient-derived pancreatic cancer or autochthonous genetically-engineered mouse models can serve as better models for more clinically-relevant testing. Similar studies can be done for lung cancer.

In conclusion, we have developed and established the initial safety and efficacy of a novel albumin-SN-38 conjugate (SSH20) in pre-clinical models of pancreatic and lung cancer. Our results confirm that 1) SSH20 is effective in lung and pancreatic cancer in vitro and in vivo with high Cav-1 expression, and 2) SSH20 is more potent than irinotecan with no apparent difference in overall toxicity in mouse models. Taken together, the advantageous properties inherent to SN-38 conjugation to albumin and targeted delivery make SSH20 an attractive candidate for further preclinical and potentially clinical study.

### Example 2. SSH20 Synthesis Protocol for CDMO/cGMP Manufacturing

### Synthesis Reagents

a. SN-38, CAS #86639-52-3, MW 392.40
b. Succinic Anhydride, CAS #108-30-5, MW 100.07
c. DBU, 1,8-Diazabicyclo[5.4.0]undec-7-ene, CAS #6674-22-2, MW 152.24
d. DMF, Dimethylformamide, CAS #68-12-2
e. DCC, N,N′-Dicyclohexylcarbodiimide, CAS #538-75-0, MW 206.33
f. NHS, N-Hydroxysuccinimide, CAS #6066-82-6, MW 115.09
g. HSA, Human Serum Albumin, MW 66.4 kDa

### Synthesis Protocol, Lab Scale, ˜3.25 g of Final Product SSH20:

a. SN-38-succinate: 392 mg SN-38 and 110 mg succinic anhydride are dissolved in 6 mL of DMF at a molar ratio of 1:1.1. 100 μL DBU is then added to the solution at equimolar to SN-38. Reaction mixture is stirred overnight in dark at room temperature to form SN-38-succinate.
b. SN-38-succinate-NHS: 294 mg DCC and 127 mg NHS are added to the reaction mixture both at a molar ratio of 1:1.1 to SN-38. The reaction is stirred at room temperature in dark for 6 hours.
c. SSH20: 20 molar excess of SN-38-suc-NHS is then added dropwise to HAS solution (03 mL of 25% HSA to the above reaction, in 15 mL 50 mM HEPES buffer at pH 8.5). The conjugation is taken place at room temperate for 4 hours in dark. 2.0N sodium carbonate solution is added to the solution to maintain solution pH at 8.5.
d. Crude Purification: The reaction mixture is centrifuge at high speed (>10,000×g) to remove DCU byproduct and solid contaminants. 4× volume of ice-cold acetone is added to the supernatant and the mixture is incubated at −80° C. for 1 hour to yield protein precipitates. The protein product is isolated by centrifugation (4,000×g) for 20 minutes at 4° C., dried in a desiccator for 1 hour and re-dissolved in PBS.
e. Fine Purification: The protein solution from above is dialyzed against PBS using 10 kDa dialysis cassettes overnight to remove soluble contaminants. Dialyzed product is then filtered through 0.45 μm PES membranes for sterile filtration and removal of invisible solid contaminants. Store the final product at −20° C.

### Quality Control, Lab Scale:

a. SN-38 concentration: by UV-Vis spectrometry (360 nm) or fluorescent spectrometry (ex 360 nm, em 550 nm).
b. Protein concentration: by BCA assay with HSA as standard.
c. Conjugation #: by MALDI-TOF-MS to determine peak shift for conjugation number analysis.

### Synthesis Protocol, Engineering Batch, ˜150 g of Final Product SSH20:

a. SN-38-succinate: 18.42 g SN-38 and 5.17 g succinic anhydride are added to 300 mL DMF in a stirring tank. 4.7 mL DBU is then added to the solution and the solution is agitated at 300 rpm at room temperature for 12-18 hours.
b. SN-38-succinate-NHS: 13.82 g DCC and 5.67 g NHS are then added to the reaction mixture and the solution is agitated at 300 rpm at room temperature for 6 hours.
c. SSH20: 600 mL 25% HSA is diluted in 2400 mL 32 mM HEPES buffer at pH 8.5 (final 5% in 25 mM HEPES, pH 8.5). Next, SN-38-succinate-NHS is added to HSA solution dropwise in a stirring tank at 300 rpm at room temperature for 4 hours. pH monitoring at pH 8.5 is required.
d. Purification: DCU solid byproduct is removed by filtration/centrifugation, and the protein product in supernatant is purified by chromatography/diafiltration. Crude-purified protein product is then further purified by diafiltration/TFF against PBS. Fine protein product is concentrated using ultrafiltration and finalized through sterile filtration.

### REFERENCES

Each of the below references is hereby individually incorporated by reference herein in its entirety for all purposes.
1. Garmann, D., Warnecke, A., Kalayda, G. V., Kratz, F., and Jaehde, U. (2008). Cellular accumulation and cytotoxicity of macromolecular platinum complexes in cisplatin-resistant tumor cells. J Control Release 131, 100-106. 10.1016/j.jconrel.2008.07.017.
2. Yang, F., and Liang, H. (2015). Editorial: HSA-based drug development and drug delivery systems. Curr Pharm Des 21, 1784. 10.2174/138161282114150326101436.
3. Kratz, F. (2014). A clinical update of using albumin as a drug vehicle—A commentary. Journal of Controlled Release 190, 331-336. 10.1016/j.jconrel.2014.03.013.
4. Kratz, F., and Elsadek, B. (2012). Clinical impact of serum proteins on drug delivery. J Control Release 161, 429-445. 10.1016/j.jconrel.2011.11.028.
5. Sleep, D., Cameron, J., and Evans, L. R. (2013). Albumin as a versatile platform for drug half-life extension. Bba-Gen Subjects 1830, 5526-5534. 10.1016/j.bbagen.2013.04.023.
6. Hoogenboezem, E. N., and Duvall, C. L. (2018). Harnessing albumin as a carrier for cancer therapies. Adv Drug Deliver Rev 130, 73-89. 10.1016/j.addr.2018.07.011.
7. Kobayashi, H., Watanabe, R., and Choyke, P. L. (2014). Improving Conventional Enhanced Permeability and Retention (EPR) Effects; What Is the Appropriate Target? Theranostics 4, 81-89. 10.7150/thno.7193.
8. Ojha, T., Pathak, V., Shi, Y., Hennink, W. E., Moonen, C. T. W., Storm, G., Kiessling, F., and Lammers, T. (2017). Pharmacological and physical vessel modulation strategies to improve EPR-mediated drug targeting to tumors. Adv Drug Deliv Rev 119, 44-60. 10.1016/j.addr.2017.07.007.
9. Golombek, S. K., May, J. N., Theek, B., Appold, L., Drude, N., Kiessling, F., and Lammers, T. (2018). Tumor targeting via EPR: Strategies to enhance patient responses. Adv Drug Deliver Rev 130, 17-38. 10.1016/j.addr.2018.07.007.
10. Mo, Y., Barnett, M. E., Takemoto, D., Davidson, H., and Kompella, U. B. (2007).

Human serum albumin nanoparticles for efficient delivery of Cu, Zn superoxide dismutase gene. Mol Vis 13, 746-757.
11. Chatterjee, M., Ben-Josef, E., Robb, R., Vedaie, M., Seum, S., Thirumoorthy, K., Palanichamy, K., Harbrecht, M., Chakravarti, A., and Williams, T. M. (2017). Caveolae-Mediated Endocytosis Is Critical for Albumin Cellular Uptake and Response to Albumin-Bound Chemotherapy. Cancer Research 77, 5925-5937. 10.1158/0008-5472.Can-17-0604.
12. Williams, T. M., and Lisanti, M. P. (2004). The Caveolin genes: from cell biology to medicine. Ann Med 36, 584-595. 10.1080/07853890410018899.
13. Sunaga, N., Miyajima, K., Suzuki, M., Sato, M., White, M. A., Ramirez, R. D., Shay, J. W., Gazdar, A. F., and Minna, J. D. (2004). Different roles for caveolin-1 in the development of non-small cell lung cancer versus small cell lung cancer. Cancer Res 64, 4277-4285. 10.1158/0008-5472.CAN-03-3941.
14. Chatterjee, M., Ben-Josef, E., Thomas, D. G., Morgan, M. A., Zalupski, M. M., Khan, G., Robinson, C. A., Griffith, K. A., Chen, C. S., Ludwig, T., et al. (2015). Caveolin-1 is Associated with Tumor Progression and Confers a Multi-Modality Resistance Phenotype in Pancreatic Cancer. Sci Rep-Uk 5. ARTN 1086710.1038/srep10867.
15. Yu, H. X., Shen, H. L., Zhang, Y., Zhong, F., Liu, Y. K., Qin, L X., and Yang, P. Y. (2014). CAV1 Promotes HCC Cell Progression and Metastasis through Wnt/beta-Catenin Pathway. Plos One 9. ARTN e10645110.1371journal.pone.0106451.
16. Kato, K., Hida, Y., Miyamoto, M., Hashida, H., Shinohara, T., Itoh, T., Okushiba, S., Kondo, S., and Katoh, H. (2002). Overexpression of caveolin-1 in esophageal squamous cell carcinoma correlates with lymph node metastasis and pathologic stage. Cancer 94, 929-933. DOI 10.1002/cncr.10329.
17. Kim, Y. J., Kim, J. H., Kim, O., Ahn, E. J., Oh, S. J., Akanda, M. R., Oh, I. J., Jung, S., Kim, K. K., Lee, J. H., et al. (2019). Caveolin-1 enhances brain metastasis of non-small cell lung cancer, potentially in association with the epithelial-mesenchymal transition marker SNAIL. Cancer Cell Int 19, 171. 10.1186/s12935-019-0892-0.
18. Bailly, C. (2019). Irinotecan: 25 years of cancer treatment. Pharmacol Res 148. ARTN 10439810.1016/j.phrs.2019.104398.
19. Ueno, M., Nonaka, S., Yamazaki, R., Deguchi, N., and Murai, M. (2002). SN-38 induces cell cycle arrest and apoptosis in human testicular cancer. Eur Urol 42, 390-397. Pii S0302-2838(02)00321-4Doi 10.1016/S0302-2838(02)00321-4.
20. Maurya, D. K., Ayuzawa, R., Doi, C., Troyer, D., and Tamura, M. (2011). Topoisomerase I Inhibitor SN-38 Effectively Attenuates Growth of Human Non-Small-Cell Lung Cancer Cell Lines In Vitro and In Vivo. J Environ Pathol Tox 30, 1-10.
21. Tamura, N., Hirano, K., Kishino, K., Hashimoto, K., Amano, O., Shimada, J., and Sakagami, H. (2012). Analysis of Type of Cell Death Induced by Topoisomerase Inhibitor SN-38 in Human Oral Squamous Cell Carcinoma Cell Lines. Anticancer Res 32, 4823-4832.
22. Rothenberg, M. L. (1997). Topoisomerase I inhibitors: review and update. Ann Oncol 8, 837-855. 10.1023/a:1008270717294.
23. Pommier, Y. (2006). Topoisomerase I inhibitors: camptothecins and beyond. Nat Rev Cancer 6, 789-802. 10.1038/nrc1977.
24. Salmanpour, M., Yousefi, G., Samani, S. M., Mohammadi, S., Anbardar, M. H., and Tamaddon, A. (2019). Nanoparticulate delivery of irinotecan active metabolite (SN38) in murine colorectal carcinoma through conjugation to poly (2-ethyl 2-oxazoline)-b-poly (L-glutamic acid) double hydrophilic copolymer. Eur J Pharm Sci 136, 104941. 10.1016/j.ejps.2019.05.019.
25. Tsuchihashi, Y., Abe, S., Miyamoto, L., Tsunematsu, H., Izumi, T., Hatano, A., Okuno, H., Yamane, M., Yasuoka, T., Ikeda, Y., et al. (2020). Novel Hydrophilic Camptothecin Derivatives Conjugated to Branched Glycerol Trimer Suppress Tumor Growth without Causing Diarrhea in Murine Xenograft Models of Human Lung Cancer. Mol Pharm 17, 1049-1058. 10.1021/acs.molpharmaceut.9b00249.
26. Carter, D. C., and Ho, J. X. (1994). Structure of serum albumin. Adv Protein Chem 45, 153-203. 10.1016/s0065-3233(08)60640-3.
27. Larsen, M. T., Kuhlmann, M., Hvam, M. L., and Howard, K. A. (2016). Albumin-based drug delivery: harnessing nature to cure disease. Mol Cell Ther 4, 3. 10.1186/s40591-016-0048-8.
28. Von Hoff, D. D., Ervin, T., Arena, F. P., Chiorean, E. G., Infante, J., Moore, M., Seay, T., Tjulandin, S. A., Ma, W. W., Saleh, M. N., et al. (2013). Increased survival in pancreatic cancer with nab-paclitaxel plus gemcitabine. N Engl J Med 369, 1691-1703. 10.1056/NEJMoa 1304369.
29. Socinski, M. A., Bondarenko, I., Karaseva, N. A., Makhson, A. M., Vynnychenko, I., Okamoto, I., Hon, J. K., Hirsh, V., Bhar, P., Zhang, H., et al. (2012). Weekly nab-Paclitaxel in Combination With Carboplatin Versus Solvent-Based Paclitaxel Plus Carboplatin as First-Line Therapy in Patients With Advanced Non-Small-Cell Lung Cancer: Final Results of a Phase III Trial. J Clin Oncol 30, 2055-2062. 10.1200/Jco.2011.39.5848.
30. Shen, Z., Wei, W., Zhao, Y. J., Ma, G. H., Dobashi, T., Maki, Y., Su, Z. G., and Wan, J. P. (2008). Thermosensitive polymer-conjugated albumin nanospheres as thermal targeting anti-cancer drug carrier. European Journal of Pharmaceutical Sciences 35, 271-282. 10.1016/j.ejps.2008.07.006.
31. Choi, S. H., Byeon, H. J., Choi, J. S., Thao, L., Kim, I., Lee, E. S., Shin, B. S., Lee, K. C., and Youn, Y. S. (2015). Inhalable self-assembled albumin nanoparticles for treating drug-resistant lung cancer. J Control Release/97, 199-207. 10.1016/j.jconrel.2014.11.008.
32. Sepehri, N., Rouhani, H., Ghanbarpour, A. R., Gharghabi, M., Tavassolian, F., Amini, M., Ostad, S. N., Ghahremani, M. H., and Dinarvand, R. (2014). Human serum albumin conjugates of 7-ethyl-10-hydroxycamptothecin (SN38) for cancer treatment. Biomed Res Int 2014, 963507. 10.1155/2014/963507.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. An albumin conjugate comprising an albumin polypeptide covalently bound to one or more groups of Formula I: 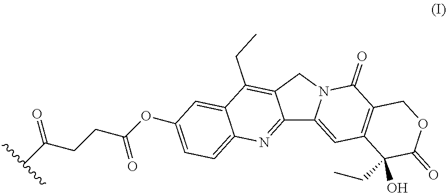 [SMILES: *C(=O)CCC(=O)Oc1ccc2nc3c(c(CC)c2c1)Cn1c-3cc2c(c1=O)COC(=O)[C@]2(O)CC]
wherein [symbol] shows the point of attachment to the albumin polypeptide.

2. The albumin conjugate of claim 1, wherein the albumin polypeptide is covalently bound to from 3 to 20 groups of Formula I.

3. The albumin conjugate of claim 1 or 2, wherein the albumin polypeptide is covalently bound to 7 or 8 groups of Formula I.

4. The albumin conjugate of any one of claims 1-3, wherein the albumin polypeptide comprises one or more lysine residues.

5. The albumin conjugate of any one of claims 1-3, wherein the albumin polypeptide comprises at least two, three, four, five, ten, or fifteen lysine residues.

6. The albumin conjugate of any one of claims 1-3, wherein the albumin polypeptide comprises human serum albumin.

7. The albumin conjugate of any one of claims 1-3, wherein the albumin polypeptide comprises bovine serum albumin.

8. The albumin conjugate of any one of claims 1-3, wherein the albumin polypeptide is a polypeptide comprising an amino acid sequence which is at least 70% identical to SEQ ID NO: 1.

9. The albumin conjugate of claim 8, wherein the albumin polypeptide is a polypeptide comprising an amino acid sequence which is at least 75%, at least 80%, at least 85%, at least 90%, at least 95%, at least 98%, or at least 99% identical to SEQ ID NO. 1.

10. The albumin conjugate of claim 8 or 9, wherein the albumin polypeptide is a polypeptide comprising SEQ ID NO: 1.

11. A pharmaceutical composition comprising a therapeutically effective amount of the albumin conjugate of any one of claims 1-10 and a pharmaceutically acceptable carrier or excipient.

12. A method of treating a cancer in a subject in need thereof comprising administering a therapeutically effective amount of an albumin conjugate of any one of claims 1-10 or a pharmaceutical composition of claim 11.

13. The method of claim 12, wherein the cancer expresses caveolin 1 (Cav-1).

14. The method of claim 12 or claim 13, wherein the cancer expresses an increased amount of Cav-1 compared to a control, wherein the control comprises a non-cancerous cell of a same tissue type as the cancer.

15. A method of treating a cancer in a subject in need thereof comprising:
a) determining whether the cancer expresses caveolin 1 (Cav-1); and
b) if the cancer is determined to express Cav-1 in a), administering a therapeutically effective amount of an albumin conjugate of any one of claims 1-10 or a pharmaceutical composition of claim 11.

16. The method of claim 15, wherein determining whether the cancer expresses Cav-1 comprises determining a level of Cav-1 in a sample from the cancer.

17. The method of claim 16, wherein determining the level of Cav-1 comprises determining a level of Cav-1 polypeptide expression or functionality in the sample.

18. The method of claim 16, wherein determining the level of Cav-1 comprises determining a level of Cav-1 polynucleotide expression or functionality in the sample.

19. A method of treating a cancer in a subject in need thereof comprising:
a) determining whether the cancer expresses an increased amount of caveolin 1 (Cav-1) compared to a control, wherein the control comprises a non-cancerous cell of a same tissue type as the cancer; and
b) if the cancer is determined to express an increased amount of Cav-1 in a), administering a therapeutically effective amount of an albumin conjugate of any one of claims 1-10 or a pharmaceutical composition of claim 11.

20. The method of claim 19, wherein determining whether the cancer expresses an increased amount of Cav-1 compared to the control comprises comparing a level of Cav-1 in a sample from the cancer to a level of Cav-1 in a sample from the control.

21. The method of claim 19, wherein comparing a level of Cav-1 comprises comparing a level of Cav-1 polypeptide expression or functionality in the sample from the cancer to a level of Cav-1 polypeptide expression or functionality in the sample from the control.

22. The method of claim 19, wherein comparing a level of Cav-1 comprises comparing a level of Cav-1 polynucleotide expression or functionality in the sample from the cancer to a level of Cav-1 polynucleotide expression or functionality in the sample from the control.

23. The method of any one of claims 12-22, wherein the cancer is selected from melanoma, mesothelioma, a sarcoma, pancreatic, lung, breast, head and neck, urothelial, bladder, kidney, esophageal, and prostate cancer.

24. The method of any one of claims 12-23, wherein the albumin conjugate or pharmaceutical composition are administered in combination or alternation with one or more additional anticancer agents.

25. An albumin conjugate of any one of claims 1-10 or a pharmaceutical composition of claim 11 for use in treating a cancer in a subject in need thereof.

26. The albumin conjugate of claim 25, wherein the cancer expresses caveolin 1 (Cav-1).

27. The albumin of claim 25 or claim 26, wherein the cancer expresses an increased amount of Cav-1 compared to a control, wherein the control comprises a non-cancerous cell of a same tissue type as the cancer.

28. The albumin conjugate of any one of claims 25-27, wherein the cancer is selected from melanoma, mesothelioma, a sarcoma, pancreatic, lung, breast, head and neck, urothelial, bladder, kidney, esophageal, and prostate cancer.

29. Use of an albumin conjugate of any one of claims 1-10 in the manufacture of a medicament for treating a cancer in a subject in need thereof.

30. The albumin conjugate of claim 29, wherein the cancer expresses caveolin 1 (Cav-1).

31. The albumin of claim 29 or claim 30, wherein the cancer expresses an increased amount of Cav-1 compared to a control, wherein the control comprises a non-cancerous cell of a same tissue type as the cancer.

32. The albumin conjugate of any one of claims 29-31, wherein the cancer is selected from melanoma, mesothelioma, a sarcoma, pancreatic, lung, breast, head and neck, urothelial, bladder, kidney, esophageal, and prostate cancer.


SEQUENCE LISTING (2 sequences)

SEQ ID NO: 1 - protein, 609 aa, synthetic construct [sequence omitted]

SEQ ID NO: 2 - DNA, 2055 bp, synthetic construct
  source 1..2055: mol_type = other DNA
agcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgggagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcttgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctgttagttcgttacaccaagaaagtacccgaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctgcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct